(19) United States
(12) Patent Application Publication
(10) Pub. No.: US 2019/0367871 A1
(43) Pub. Date: Dec. 5, 2019

(54) EMBRYONIC STEM CELL MEDIA AND CULTURE

(71) Applicant: YISSUM RESEARCH DEVELOPMENT COMPANY OF THE HEBREW UNIVERSITY OF JERUSALEM LTD, Jerusalem (IL)

(72) Inventor: Nissim BENVENISTY, Jerusalem (IL)

(21) Appl. No.: 16/424,841

(22) Filed: May 29, 2019

Related U.S. Application Data
(60) Provisional application No. 62/677,236, filed on May 29, 2018.

(51) Int. Cl. C12N 5/0735; C12N 5/074; C12N 15/11; C12N 9/22

(52) U.S. Cl. CPC C12N 5/0606; C12N 5/0696; C12N 15/11; C12N 2800/80; C12N 2501/105; C12N 2501/48; C12N 2310/20; C12N 9/22

(57) ABSTRACT

Methods of culturing pluripotent stem cells (PSCs) and maintaining or reducing p53 mutation is those cells are provided. Media for PSC culture is also provided.

### CROSS REFERENCE TO RELATED APPLICATIONS

This application is a U.S. Patent Application which claims the benefit of priority of U.S. Provisional Patent Application No. 62/677,236, filed May 29, 2018, the contents of which are all incorporated herein by reference in their entirety.

### FIELD OF INVENTION

The present invention is in the field of embryonic stem cell culture.

### BACKGROUND OF THE INVENTION

Haploid cells allow genetic screening through the generation of a highly enriched hemizygous mutant library, owing to the single set of chromosomes in these cells. While much of the previous work on haploid genetics has been carried out in unicellular organisms, recent developments have made it possible to extend this field into mammalian cells.

Haploid human ESCs (hESCs) were recently isolated. These cells exhibit human pluripotent stem cell (PSC) features in their colony morphology, alkaline phosphatase activity, gene expression signatures and epigenetic profiles. Interestingly, haploid hESCs can differentiate into haploid somatic cells in vitro and in vivo, generating cell types representative of the three embryonic germ layers. Haploid hESCs can be grown in standard culture conditions for over 30 passages while retaining a normal haploid karyotype. Therefore, haploid hESCs provide an efficient screening platform to address questions regarding pluripotency on a genome-wide level.

Haploid mammalian cells have recently been used for loss-of-function genetic screens. Initial loss-of-function screens in humans have utilized a near-haploid leukemic cell line. This transformed cancer cell line has been previously used to identify the host factors used by human pathogens and more recently, it has been utilized for a genome-wide loss-of-function screen for identifying essential genes in the human genome and for studying synthetic lethality between different genes. However, because these cells are not embryonic stem cells, the essentialome of ESCs is as yet unknown. Further, media and methods for improved ESC culture are greatly needed.

### SUMMARY OF THE INVENTION

The present invention provides methods for culturing pluripotent stem cells (PSCs) and for maintaining or reducing p53 mutation in those cells. Media for culturing PSCs is also provided.

According to a first aspect, there is provided a method of maintaining or reducing the frequency of p53 mutation in a population of pluripotent stem cells (PSCs), the method comprising at least one of:
a. activating mTOR signaling in the population;
b. inhibiting Bax expression, function or both in the population;
c. inhibiting phorbol-12-myristate-13-acetate-induced protein 1 (Noxa), expression, function or both in the population;
d. inhibiting p53 apoptosis effector related to PMP22 (PERP) expression, function or both in the population; and
e. inhibiting zinc finger matrin-type 3 (PAG608) expression, function or both in the population;
   thereby maintaining or reducing the frequency of p53 mutation in a population of PSC.

According to another aspect, there is provided a method of culturing a population of PSCs, the method comprising performing at least two of the following to the population in culture:
a. activating mTOR signaling in the population;
b. inhibiting Bax expression, function or both in the population;
c. inhibiting phorbol-12-myristate-13-acetate-induced protein 1 expression (Noxa), function or both in the population;
d. inhibiting p53 apoptosis effector related to PMP22 (PERP) expression, function or both in the population; and
e. inhibiting zinc finger matrin-type 3 (PAG608) expression, function or both in the population;
   thereby culturing a population of PSC.

According to another aspect, there is provided a PSC media comprising at least one of:
a. a Bax inhibitor;
b. a Noxa inhibitor;
c. a PERP inhibitor; and
d. a PAG608 inhibitor.

According to some embodiments, the PSCs are selected from embryonic stem cells (ESCs) and induced pluripotent stem cells (iPSCs).

According to some embodiments, the PSCs are human PSCs.

According to some embodiments, the inhibiting comprises supplementing media comprising the hESCs with an inhibitor and the activating comprises supplementing media comprising the hESCs with an mTOR activator.

According to some embodiments, the activator is a direct activator, the inhibitor is a direct inhibitor, or both.

According to some embodiments, the activating comprises inhibiting expression, function or both of an inhibitory protein of the mTOR pathway selected from PTEN, TSC1 and TSC2.

According to some embodiments, the method of the invention does not comprise differentiation of the PSCs.

According to some embodiments, the method of the invention does not increase differentiation of the PSCs as compared to control PSCs.

According to some embodiments, the method of the invention does not decrease apoptosis in the population.

According to some embodiments, the method of the invention increases the growth rate of the population.

According to some embodiments, the method of the invention further comprises using the population of PSCs in an assay requiring wild type p53 expressing cells.

According to some embodiments, the culturing
a. does not comprises differentiation of the hESCs;
b. does not decrease apoptosis in the population;
c. increases the growth rate of the hESCs; or
d. a combination thereof.

According to some embodiments, the media of the invention further comprises an mTOR activator.

According to some embodiments, the mTOR activator is insulin-like growth factor 1 (IGF1).

According to some embodiments, the inhibitor
a. inhibits mRNA stability, mRNA translation or protein function; or
b. is a direct inhibitor of at least one of Bax, Noxa, PERP and PAG608.

According to some embodiments, the PSC is selected from an ESC and an iPSC, optionally wherein the PSC is a human PSC.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1E: Establishment and characterization of a genome-wide CRISPR-Cas9 screen in haploid hPSCs. (FIG. 1A) Schematic illustrating the generation of the mutant library. (FIG. 1B) Distribution of the number of gRNA reads/gene at indicated time points after gRNA infection. (FIG. 1C) Top, schematic representation of the genomic loci of two pluripotency-associated genes (POU5F1 and PRDM14) and their neighboring genes expressed in hESCs; middle, mean±s.e.m. of gRNA reads/gene over time in culture (n=20 gRNAs (two biological replicates of 10 independent gRNAs per gene). Bottom, expression levels of the genes in ESCs and 14 somatic cell types (from left to right: skin, brain, heart, liver, skeletal muscle, pancreas, lung, stomach, blood, small intestine, kidney, adipose, transformed fibroblasts and transformed lymphocytes). (FIG. 1D) Percentages of essential genes (red) and growth-restricting genes (blue). Genes with FDR less than 0.05 are regarded as significant (Kolmogorov-Smirnov test (KS-test), n=20 gRNAs). (FIG. 1E) Chromosomal distribution of essential genes (red lines) and all other genes targeted in the library (grey lines).

FIGS. 2A-2F: Analysis of cell-essential genes. (FIG. 2A) Distribution of cell-essential genes across cellular compartments. Essential-gene percentages of the nucleus and mitochondrion compartments were significantly increased over their representation in the library (Hypergeometric test (HG-test), n=872 genes, P=52e⁻⁶⁵ and P=32e⁻¹², respectively). (FIG. 2B) Top, fraction of essential genes within the total number of genes in each cellular compartment; bottom, ratio of growth-restricting genes over essential genes in each cellular compartment. (FIG. 2C) CRISPR score represents the average log₂ fold change in the abundance of the gRNAs of each gene between final and initial populations. Shown is the distribution of the CRISPR scores of genes associated with autosomal-recessive human disorders (blue curve) and the subset of these genes also associated with a growth retardation phenotype (red curve). (FIG. 2D) Levels of gRNA reads/gene over time in culture for Fanconi anemia-causing genes (shades of red) and tuberous sclerosis-causing genes (shades of blue). (FIGS. 2E-2F) Volcano plots representing Q-value and CRISPR score of canonical oncogenes (FIG. 2E) and tumor suppressor genes (FIG. 2F); dotted line: Q=0.05 (KS-test, n=20 gRNAs); in blue: essential genes, in red: growth-restricting genes, in dark blue: apoptosis-related genes, in dark red: genes related to genomic instability and DNA repair.

FIGS. 3A-3E: Identification and characterization of the hESC-essentialome. (FIG. 3A) Distribution of CRISPR scores of genes in hESCs. From top to bottom: all genes; hESC-expressed genes; hESC-enriched genes; nuclear hESC-enriched genes. Pie charts show the percentage of essential genes in the gene subsets represented by the red curves. (FIG. 3B) Distribution of the hESC-essentialome across cellular compartments. (FIG. 3C) Functional categorization of the hESC-essentialome. (FIG. 3D) Volcano plot representing significance and CRISPR score of hESC-enriched transcription factors; dotted line: P=0.05 (KS-test, n=20 gRNAs), in blue: essential genes, in red: growth-restricting genes. (FIG. 3E) Expression ratio between embryoid bodies (EB) and hESCs for the hESC-enriched transcription factors (TFs). TFs whose gRNAs are significantly depleted (red bar) or not depleted (blue bar) are shown (mean±s.e.m. values, two-tailed t-test, n=8 depleted hESC-enriched transcription factors, n=22 non-depleted hESC-enriched transcription factors, *P=0.027).

FIGS. 4A-4D: Analysis of hESC-essential genes on the survival and pluripotency of hESCs. (FIG. 4A) Validation of essential genes representing different functional categories in the hESC-essentialome via sgRNA-mediated knockout (upper panel) and siRNA knockdown (lower panel) in diploid hESCs. Either a Cas9-containing sgRNA-free vector or a non-targeting siRNA was used as a control. Shown are mean±s.e.m. values of the effects of each sgRNA (two-tailed t-test, n=6 biological replicates, P_{SALL4}=1.6 e⁻⁴, P_{DSCC1}=3.1e⁻⁵, P_{SEPHS1}=2.6 e⁻⁴, P_{VRTN}=1.9e⁻¹¹) and siRNA on cell growth (two-tailed t-test, n=3 biological replicates, P_{SALL4=5.5}e⁻⁵, P_{DSCC1}=1.29e⁻⁶, P_{SEPHS1}=2.32e⁻⁷, P_{VRTN}=8.9e⁻⁶). (**P<0.01, ***P<0.001 unpaired t-test). (FIG. 4B) Cell viability assay in KBM7 sgRNA-knockout lines for DSCC1 and SEPHS1 4 days after the delivery of sgRNAs and Cas9. Control lines received only Cas9 in the absence of a sgRNA (two-tailed t-test, n=3 biological replicates, P_{DSCC1}=0.001, P_{SEPHS1}=0.2). (FIG. 4C) PCA plot demonstrating the biological replicates of the transcriptome of hESCs with siRNA knockdown for SALL4, DSCC1, SEPHS1 and VRTN (n=60,675 genes). (FIG. 4D) The percentage of downregulated hESC-enriched transcripts upon siRNA knockdown of target genes, divided into different groups of fold reduction. The reduction in expression of pluripotent genes in the cells with knockdown of each of the genes was significant, as calculated by the comparison of the percentage of significantly downregulated genes in the hESC-essential genes to that in control cells (siRNA for Renilla Luciferase), (two-tailed proportion test, P_{SALL4}=0.029, P_{DSCC1}=0.008, P_{SEPHS1}=0.0006 and P_{VRTN}=0.017). e, Gene ontology analysis of upregulated genes upon siRNA knockdown of target genes (n_{SALL4}=922 genes, N_{SEPHS1}=174 genes, n_{DSCCL}=217 genes, n_{VRTN}=539 genes). When applicable, mean±s.e.m. are presented and unpaired two-tailed t-test was applied (**P<0.01; ***P<0.001).

FIG. 4E: Chart showing knockdown of either SEPHS1 or DSCC1 affected energy metabolism through oxidative phosphorylation, whereas VRTN knockdown showed a modest effect on mitosis.

FIGS. 5A-5G: Analysis of growth-restricting genes in hESCs. (FIG. 5A) Gene ontology analysis for the top 50 growth-restricting genes in hESCs (n=50 genes). (FIG. 5B) Comparison of the CRISPR scores of all genes from the current haploid hESC screen and a previous screen in the near-haploid leukemic cell line KBM7. Genes related to P53 and ROCK pathways, which are among the top growth-restricting genes in hESCs, are highlighted. (FIG. 5C) Schematic representation of the P53-mTOR pathway, highlighting the growth-restricting genes in hESCs. (FIG. 5D) Growth curves of IGF1-treated (orange) and untreated control (blue) diploid hESCs grown in conditioned medium with 1.2% KSR (n=4 biological replicates, P=1.07e⁻⁵). (FIG. 5E) Percentages of apoptotic cells (left), cells in G2/M phase (middle) and TRA-1-60-positive pluripotent cells, in control and IGF1-treated diploid hESCs (n=3 biological replicates, P=0.006). (FIG. 5F) Growth curves of untreated control (blue), IGF1-treated (orange), rapamycin-treated (purple) and rapamycin+IGF1-treated (green) diploid hESCs (n=3 biological replicates, P=0.001). (FIG. 5G) Relative cell numbers of human foreskin fibroblasts (blue), leukemic KBM7 cells (green) and diploid hESCs (red) after two days of rapamycin treatment with the indicated concentrations (n=3 biological replicates, P_{Fibroblasts}=8.1 e⁻⁵, P_{KBM7}=4.6e⁻³). When applicable, mean±s.e.m. are presented and unpaired two-tailed t-test was applied (**P<0.01; ***P<0.001).

FIGS. 6A-6J: Characterization of the selective sensitivity of hESCs to mTOR inhibition. (FIG. 6A) Dose response of Torin 1-treated human fibroblasts (pink) and three different hESC lines (shades of blue) at indicated concentrations after two days of treatment (n=3 biological replicates). (FIG. 6B) Growth curves of rapamycin- and Torin 1-treated fibroblasts (pink) and hESCs (blue). Shown are the values normalized to the untreated controls of the corresponding time points (n=3 biological replicates for each time point). (FIG. 6C) Gene ontology analysis for significantly downregulated genes after two days of rapamycin treatment of hESCs (blue, n=593 genes) and fibroblasts (pink, n=243 genes) (left inset). Also shown is the relative expression of MAPK pathway members (middle) or lipid metabolism-related genes (right) in rapamycin-treated hESCs (blue) and fibroblasts (pink) for genes expressed in both cell types (FPKM>1). (FIG. 6D) Flow cytometry analysis of apoptotic hESCs (diploid CSES9 cell line) upon two days of rapamycin treatment. Shown are representative analyses out of three biological replicates of untreated (left) and rapamycin-treated hESCs (middle). Also shown is percentage of apoptotic cells in untreated and rapamycin-treated hESCs (n=3 biological replicates, P=0.009) (right). (FIG. 6E) Expression levels of TP53 in hESCs (blue) and fibroblasts (pink) upon rapamycin treatment for indicated durations. FPKM values were normalized to the expression levels of the corresponding untreated controls in each cell type (n=3 biological replicates, P=0.02). (FIG. 6F) Cell cycle analysis of hESCs by flow cytometry upon two days of rapamycin treatment. Shown are representative analyses out of three biological replicates. (FIG. 6G) Percentages of hESCs in G1 (left) and G2/M (right) phases in untreated and rapamycin-treated hESCs (n=3 biological replicates, P_{G1}=0.004, P_{G2/M}=0.004). (FIG. 611) Expression levels of CCDN1 and CCDN2 in hESCs (blue) and fibroblasts (pink) upon two days of rapamycin treatment (n=3 biological replicates, P_{CCDN1}=0.01, P_{CCDN2}=0.01). (FIG. 6I) Analysis of the TRA-1-60⁺ hESCs after four days of rapamycin treatment. Shown are representative analyses out of three biological replicates. (FIG. 6J) Percentage of TRA-1-60⁺ hESCs in untreated and rapamycin-treated hESCs (n=3 biological replicates, P=0.02). When applicable, mean±s.e.m. are presented, unpaired two-tailed t-test was applied.

FIGS. 7A-7D: Characterization of a genome-wide CRISPR-Cas9 screen in haploid hPSCs. (FIG. 7A) Analysis of the abundance of sgRNAs within the haploid hESC population at multiple time points after the co-delivery of sgRNAs and Cas9. Shown is a correlation matrix for all time points with correlation coefficients on the right diagonal and the scatter plots on the left diagonal of the matrix (n=181,131 gRNAs). (FIG. 7B) CRISPR score formula. (FIG. 7C) Scatter plot of CRISPR scores between two replicate experiments (n=18,166 genes, r=Pearson's correlation coefficient). (FIG. 7D) Chromosomal distribution of growth-restricting genes. Shown are growth-restricting genes (blue lines) and all other genes targeted in the library (grey lines).

FIGS. 8A-8D: Comparison of essential genes across various studies. (FIG. 8A) Venn diagram showing the overlap of cell-essential genes identified in the current study in hESCs and in near-haploid KBM7 leukemic cells and in several immortalized and cancer lines from different tissues of origin. (FIG. 8B) PCA plot demonstrating the separation of genes in different studies between the current study, another that used the same sgRNA library, oen that used a transposon-mediated gene-trap mutant library and one that used two independent sgRNA libraries. (FIG. 8C) Analysis of first percentile CRISPR scores across autosomes of near-haploid KBM7 cells (in which chromosome 8 is diploid), three diploid cell lines used and haploid hESCs from the current study. Chromosome 8 is highlighted in red. Dashed lines indicate the threshold corresponding to p-value of 0.025 on either side of the distribution of first percentile CRISPR scores (two-tailed Z-test, n=22 chromosomes, *P=0.02). (FIG. 8D) −log₁₀ P-values for chromosome 8 in the distribution of the first percentile CRISPR scores across autosomes in different cell lines. In near-haploid KBM7 cells, the first percentile CRISPR score for the diploid chromosome 8 is significantly different from those of the other autosomes (marked by asterisk), suggesting more efficient loss-of-function analysis in haploid chromosomes (two-tailed Z-test, n=22 chromosomes, *P=0.02).

FIGS. 9A-9G: Analysis of cell-essential genes for their cellular compartmentalization, disease association and tumor formation. (FIG. 9A) Pie charts for each cellular compartment in hESCs demonstrating both enriched and depleted genes. (FIG. 9B) Fraction of genes that are essential and expressed in hESCs within each cellular compartment (FIG. 9C) Fraction of essential genes within the total number of genes in each cellular compartment in KBM7 cells. (FIG. 9D) Distribution of CRISPR scores of genes associated with autosomal-recessive human disorders with a growth retardation phenotype in hESCs (green curve) and in leukemic KBM7 cells (purple curve). (FIGS. 9E-9F) Volcano plots representing Q-value and CRISPR score of the canonical oncogenes (FIG. 9E) and tumor suppressor genes (FIG. 9F) that did not show significant values (see FIGS. 2E-2F, KS-test, n=20 gRNAs (two biological replicates of 10 independent gRNAs per gene)). (FIG. 9G) Heatmaps showing the comparison of CRISPR scores of three groups of genes between hESCs and the cancer cell lines: Growth-restricting tumor suppressors in hESCs that lost this feature of growth-restriction in cancer cell lines (left heatmap), essential oncogenes in hESCs that lost their essentiality in cancer cell lines (middle heatmap) and non-essential oncogenes in hESCs that gained essentiality in cancer cell lines (right heatmap). Light blue and light red boxes designate significant FDR values (shown below the CRISPR score values, positive and negative values indicate enriched and depleted gRNAs, respectively) for growth-restricting and essential genes, respectively (KS-test, n=20 gRNAs).

FIGS. 10A-10G: Analysis of the hESC-essential genes. (FIG. 10A) Scatter plot demonstrating CRISPR scores of hESC-enriched transcription factors in hESCs and KBM7 cells; in green: essential genes in KBM7 cells and hESCs, in red: essential genes only in hESCs. (FIG. 10B) Mean±s.e.m. of gRNA reads/gene over time in culture for validated genes (SALL4, DSCC1, VRTN, SEPHS1) along with their expression (normalized FPKM from RNA sequencing data) in hPSCs and 27 somatic cell types. Lymphocytes and fibroblasts are transformed cell lines. CRISPR scores and P-values appear at the bottom (KS-test, n=20 gRNAs). (FIG. 10C) Relative transcript levels (analyzed by qRT-PCR) of targeted hESC-essential genes by siRNA knockdown as compared to a non-targeting siRNA control in diploid hESCs. Cells were harvested 24 hours after transfection of siRNAs for SALL4, 48 hours for VRTN and 72 hours for DSCC1 and SEPHS1 (n=3 biological replicates, mean±s.e.m values). (FIG. 10D) Relative wild-type transcript levels (analyzed by qRT-PCR) of targeted hESC-essential genes in sgRNA knockout lines 3 days after the delivery of sgRNAs and Cas9. Control lines received only Cas9 in the absence of a sgRNA. Values represent the averages of two biological replicate experiments with three technical replicates for each. (FIG. 10E) Cell viability assay in hESC and KBM7 sgRNA-knockout lines for PIK3CA and PDIA4, two genes expressed in both cell types, 4 days after the delivery of sgRNAs and Cas9 (n=3 biological replicates for KBM7 cells, n=6 biological replicates for hESC cells, mean±s.e.m values). (FIG. 10F) Relative wild-type transcript levels (analyzed by qRT-PCR) of targeted genes in sgRNA knockout lines of KBM7 cells, 3 days after the delivery of sgRNAs and Cas9. Values represent the average of three technical replicates. (FIG. 10G) Relative wild-type transcript levels (analyzed by qRT-PCR) of targeted hESC-essential genes in sgRNA knockout lines 3 days after the delivery of sgRNAs and Cas9. Values represent the average of two biological replicate experiments with three technical replicates for each.

FIG. 11: Schematics illustrating distinct P53 target pathways. Highlighted in red are significantly enriched growth-restricting genes that were found among the top 50 genes of this group in the haploid hESC screening. Enrichment fold-changes of TP53 target genes are shown on the right. Numbers indicate different P53 target pathways, red and black bars show high and low-ranking growth-restricting genes, respectively.

FIGS. 12A-12L: Selective sensitivity of hESCs to IGF1-mTOR pathway. (FIGS. 12A-12D) Growth curves of IGF1-treated (red) vs. control (blue) diploid hESCs grown in mTESR1 (FIG. 12A), conditioned medium with 12% KSR (standard) (FIG. 12B), conditioned medium with 6% KSR (FIG. 12C), conditioned medium with 3% KSR (FIG. 12D) (n=4 biologically independent samples). Mean±s.e.m. are presented and unpaired two-tailed t-test was applied (P_{mTeSR1}=0.01, P_{12% KSR}=2.7e⁻⁴, P_{6% KSR}≤8.4 e⁻⁴, P_{3% KSR}=3.6 e⁻⁴, *P<0.05; ***P<0.001). (FIGS. 12E-12G) Gating strategy for flow cytometry analysis of apoptotic cells (FIG. 12E, Annexin-V staining), cell-cycle profile (FIG. 12F, Propidium Iodide staining) and TRA-1-60⁺ pluripotent cells (FIG. 12G, TRA-1-60-staining). A minimum of 30,000 cells were analyzed for each sample. (FIGS. 12H-12I) Western blots demonstrating phosphorylated levels of mTORC1 target, 4E-BP1 and mTORC2 target AKT (top panels) upon treatment of hESCs with rapamycin and Torin 1 for short- (FIG. 12H) and long-term (FIG. 12I) treatments. To enable incubation with three primary antibodies separately, nitrocellulose membrane was cut where the dashed lines were indicated. Quantification of the western blots are shown in the bottom panels (n=3 biological replicates, mean±s.d. values). As shown, rapamycin inhibits the activity of mTORC1 but not that of mTORC2 in hESCs in both short- and long-term treatments, whereas Torin 1 inhibits both complexes. (FIG. 12J-12K) Growth curves of rapamycin- or Torin 1-treated diploid hESC lines, REX1-GFP WA09 (FIG. 12J) and CSES9 (FIG. 12K), over the course of four days (n=3 biological replicates, mean±s.e.m values). Shown are the values normalized to the untreated controls of the corresponding time points. (FIG. 12L) Gene ontology analysis for the significantly down-regulated transcripts after four days of rapamycin treatment in hESCs (CSES9, blue) and in human foreskin fibroblasts (pink) (n=3 biological replicates).

FIG. 13: A line graph of the relative growth of hESCs over time grown in the presence of varying concentrations of Bax inhibitor.

### DETAILED DESCRIPTION OF THE INVENTION

The present invention, in some embodiments, provides methods and media for culturing pluripotent stem cells.

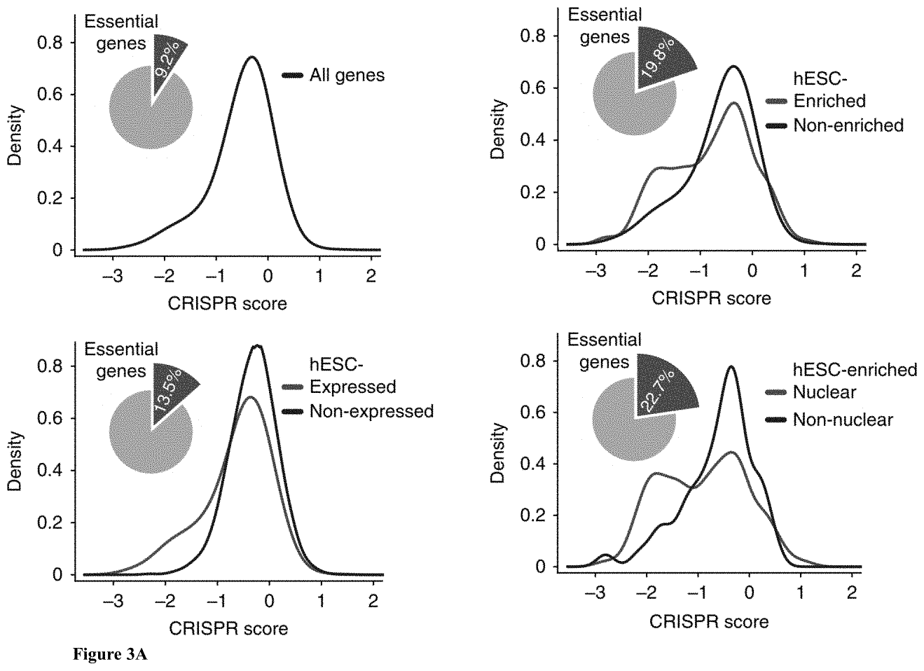

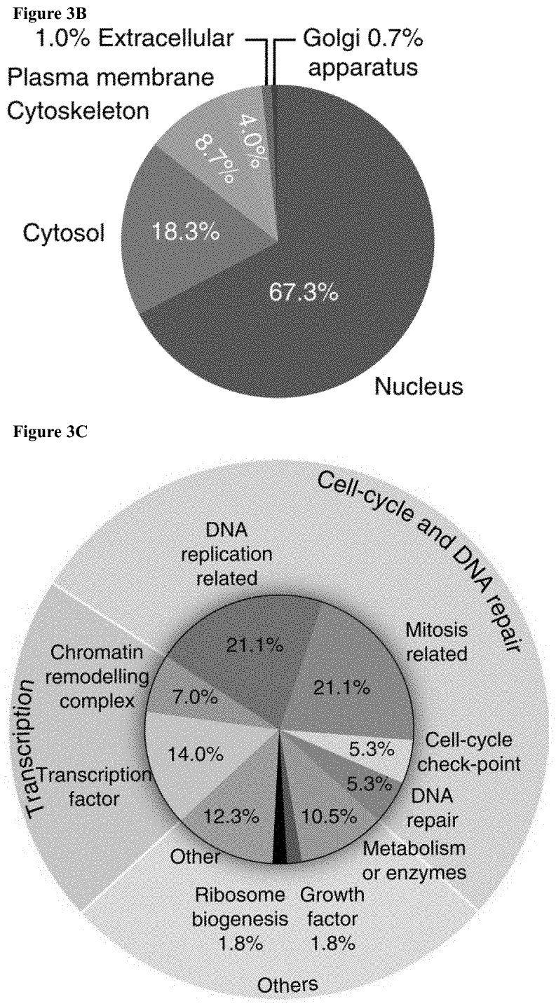

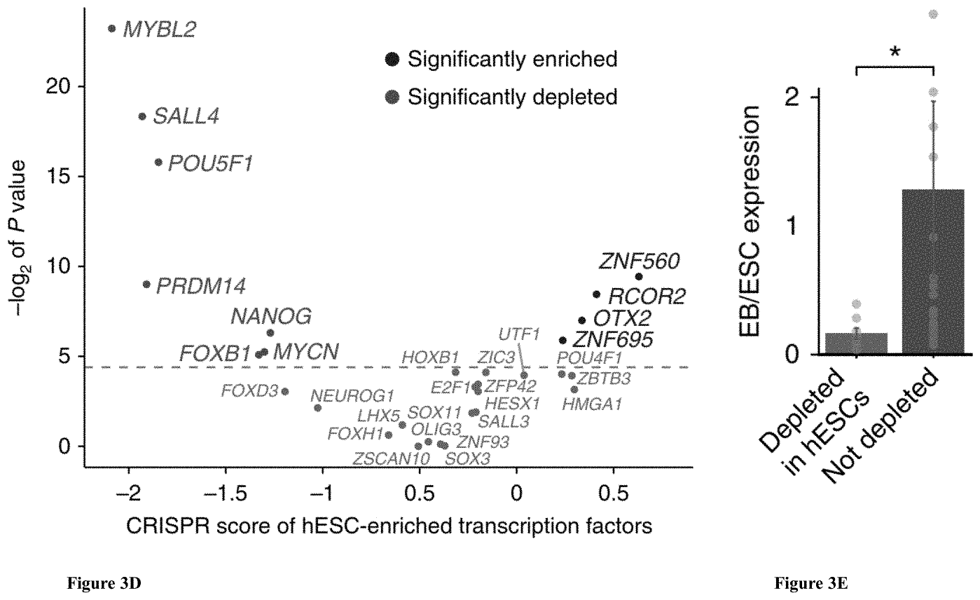

Classical descriptions of pluripotency highlight a complex TF network that governs the gene expression profile of this highly versatile cell state. Previous gene expression studies suggested several TFs as markers of pluripotency. When combined with such gene expression analyses, the essential gene screen in hESCs provided herein reveals that the majority of these pluripotency-associated TFs are dispensable for the growth and survival of hESCs (see FIG. 3D). Seven essential TFs with enriched expression in hESCs were identified. Two of these factors were the oncogenic factors, MYBL2 and MYCN. Another oncogenic factor, c-Myc, has been used to increase the efficiency of induced pluripotency. Thus, unexpectedly, at least one of MYBL2 and MYCN can replace c-Myc in the reprogramming factor cocktails to yield more authentic induced pluripotent stem cells.

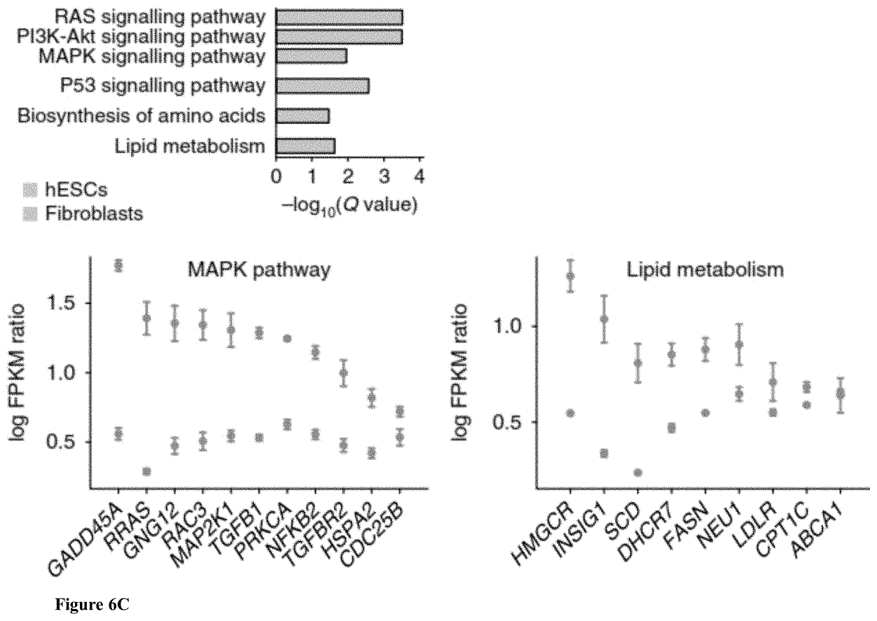

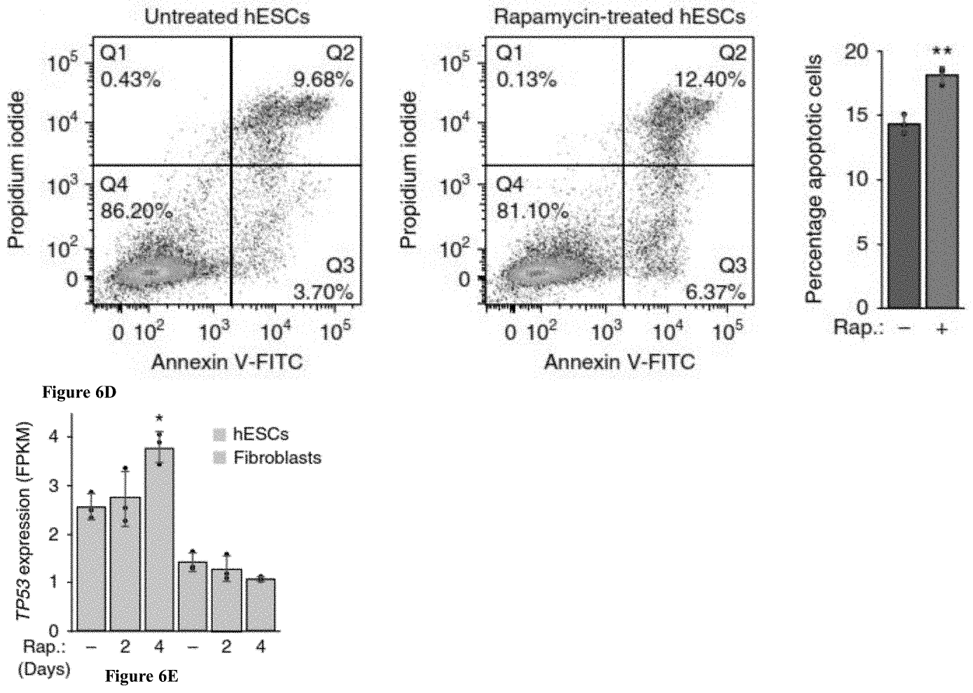

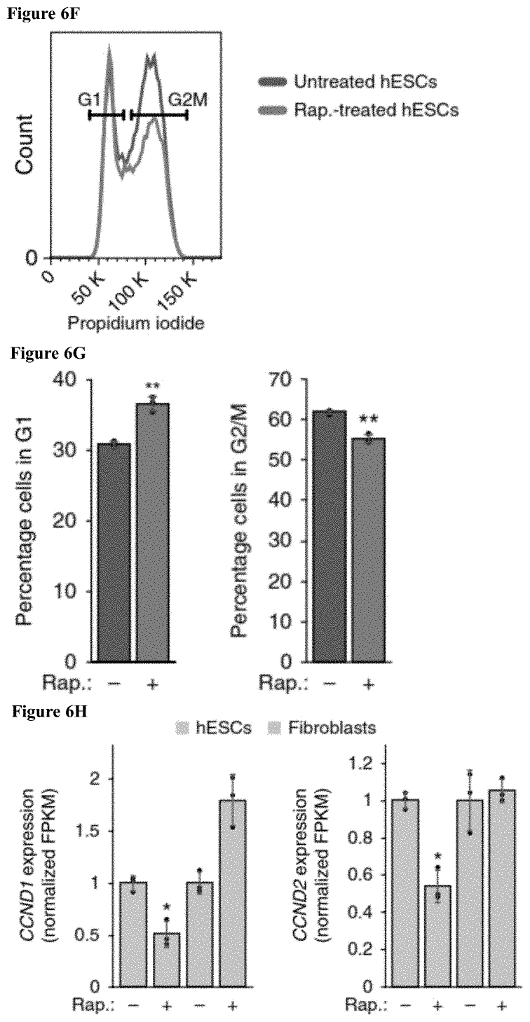

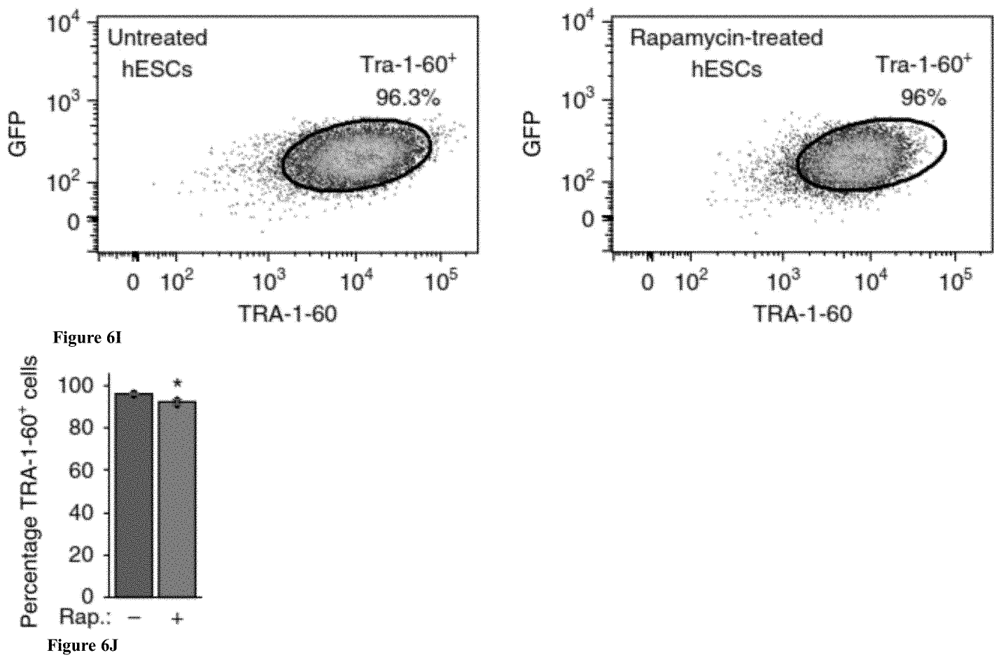

The finding that the growth of hESCs is regulated in a cell-type-selective manner by the P53-mTOR pathway is significant in light of the fact that the most prevalent mutations among hPSC lines occur in TP53. The invention is based on the surprising finding that the selective advantage of TP53 mutations might be overridden by providing chemical mTOR-activators or inhibiting other downstream effectors of p53 in culture and hence preventing the overgrowth of TP53 mutants. Conversely, cell-type selective sensitivity to mTORC1 inhibition may be used to eliminate undesired pluripotent cells from terminally differentiated cultures. mTORC1 inhibition in hESCs has been suggested to cause endoderm and mesoderm differentiation. Although a modest downregulation of the surface expression of TRA-1-60 by mTORC1 inhibition was identified, the most profound effects of this inhibition were on the levels of apoptosis and cell-cycle regulation (see FIGS. 6D-6J).

By a first aspect, there is provided, a method of culturing a population of cells, the method comprising activating mTOR signaling in the population.

By another aspect, there is provided, a method of culturing a population of cells, the method comprising inhibiting Bax in the population.

By another aspect, there is provided, a method of culturing a population of cells, the method comprising inhibiting phorbol-12-myristate-13-acetate-induced protein 1 (Noxa) in the population.

By another aspect, there is provided, a method of culturing a population of cells, the method comprising inhibiting p53 apoptosis effector related to PMP22 (PERP in the population.

By another aspect, there is provided, a method of culturing a population of cells, the method comprising inhibiting zinc finger matrin-type 3 (PAG608) in the population.

By another aspect, there is provided, a method of culturing a population of cells, the method comprising at least one of:
a. activating mTOR signaling in the population;
b. inhibiting Bax in the population;
c. inhibiting phorbol-12-myristate-13-acetate-induced protein 1 (Noxa), in the population;
d. inhibiting p53 apoptosis effector related to PMP22 (PERP) in the population; and
e. inhibiting zinc finger matrin-type 3 (PAG608) in the population,
   thereby culturing a population of cells.

In some embodiments, the cells are stem cells. In some embodiments, the stem cells are pluripotent stem cells (PSCs). In some embodiments, the PSCs are induced PSCs (iPSCs). In some embodiments, the PSCs are embryonic stem cells (ESCs). In some embodiments, the PSCs are selected from ESCs and iPSCs. In some embodiments, the cells are mammalian cells. In some embodiments, the mammal is a rodent. In some embodiments, the rodent is a mouse. In some embodiments, the mammal is a human. In some embodiments, the cells are human ESCs.

In some embodiments, the culturing comprises maintaining and/or reducing p53 mutation in the population.

By another aspect, there is provided a method or maintaining and/or reducing p53 mutation in a population of cells, the method comprising at least one of:
a. activating mTOR signaling in the population;
b. inhibiting Bax in the population;
c. inhibiting phorbol-12-myristate-13-acetate-induced protein 1 (Noxa), in the population;
d. inhibiting p53 apoptosis effector related to PMP22 (PERP) in the population; and

e. inhibiting zinc finger matrin-type 3 (PAG608) in the population, thereby maintaining and/or reducing p53 mutation in a population of cells.

The term “pluripotent stem cell” refers to cells capable of differentiating or being differentiated by means known to one ordinary in the art, into cells of any lineage. The term “embryonic stem cell” refers to stem cells derived from the undifferentiated inner mass of an embryo. Such cells are pluripotent, and capable of differentiating, or being differentiated by means known to one ordinary in the art, into cells of any lineage. In order for a ESC to be considered undifferentiated, it must continue to express stem cell markers or not express markers of differentiated cells.

The term PSC refers to pluripotent stem cells regardless of their derivation, the term PSC encompasses the terms ESC and iPSC, as well as the term embryonic germ stem cells (EGSC), which are another example of a PSC. PSCs may be in the form of an established cell line, they may be obtained directly from primary embryonic tissue, or they may be derived from a somatic cell. PSCs can be target cells of the methods described herein.

ESC lines are listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, HSF-6 (University of California at San Francisco); and HI, H7, H9, HI 3, H14 (Wisconsin Alumni Research Foundation (WiCell Research Institute)). Stem cells of interest also include embryonic stem cells from other primates, such as Rhesus stem cells and marmoset stem cells. The stem cells may be obtained from any mammalian species, e.g. human, equine, bovine, porcine, canine, feline, rodent, e.g. mice, rats, hamster, primate, etc. (Thomson et al. (1998) Science 282:1145; Thomson et al. (1995) Proc. Natl. Acad. Sci USA 92:7844; Thomson et al. (1996) Biol. Reprod. 55:254; Shamblott et al., Proc. Natl. Acad. Sci. USA 95:13726, 1998). In culture, ESCs typically grow as flat colonies with large nucleo-cytoplasmic ratios, defined borders and prominent nucleoli. In addition, ESCs express SSEA-3, SSEA-4, TRA-1-60, TRA-1-81, and Alkaline Phosphatase, but not SSEA-1. Examples of methods of generating and characterizing ESCs may be found in, for example, U.S. Pat. Nos. 7,029,913, 5,843,780, and 6,200,806. Methods for proliferating hESCs in the undifferentiated form are described in WO 99/20741, WO 01/51616, and WO 03/020920.

By “embryonic germ stem cell” (EGSC) or “embryonic germ cell” or “EG cell” is meant a PSC that is derived from germ cells and/or germ cell progenitors, e.g. primordial germ cells, i.e. those that would become sperm and eggs. Embryonic germ cells (EG cells) are thought to have properties similar to embryonic stem cells as described above. Examples of methods of generating and characterizing EG cells may be found in, for example, U.S. Pat. No. 7,153,684; Matsui, Y., et al., (1992) Cell 70:841; Shamblott, M., et al. (2001) Proc. Natl. Acad. Sci. USA 98: 113; Shamblott, M., et al. (1998) Proc. Natl. Acad. Sci. USA, 95:13726; and Koshimizu, U., et al. (1996) Development, 122:1235.

By “induced pluripotent stem cell” or “iPSC” it is meant a PSC that is derived from a cell that is not a PSC (i.e., from a cell this is differentiated relative to a PSC). iPSCs can be derived from multiple different cell types, including terminally differentiated cells. iPSCs have an ES cell-like morphology, growing as flat colonies with large nucleo-cytoplasmic ratios, defined borders and prominent nuclei. In addition, iPSCs express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26al, TERT, and zfp42. Examples of methods of generating and characterizing iPSCs may be found in, for example, U.S. Patent Publication Nos. US20090047263, US20090068742, US20090191159, US20090227032, US20090246875, and US20090304646. Generally, to generate iPSCs, somatic cells are provided with reprogramming factors (e.g. Oct4, SOX2, KLF4, MYC, Nanog, Lin28, etc.) known in the art to reprogram the somatic cells to become pluripotent stem cells.

By “somatic cell” it is meant any cell in an organism that, in the absence of experimental manipulation, does not ordinarily give rise to all types of cells in an organism. In other words, somatic cells are cells that have differentiated sufficiently that they will not naturally generate cells of all three germ layers of the body, i.e. ectoderm, mesoderm and endoderm. For example, somatic cells would include both neurons and neural progenitors, the latter of which may be able to naturally give rise to all or some cell types of the central nervous system but cannot give rise to cells of the mesoderm or endoderm lineages.

In some embodiments, p53 mutation is the level of p53 mutation in the population. In some embodiments, p53 mutation is the frequency of p53 mutation in the population. In some embodiments, p53 mutation is the prevelance of p53 mutation in the population. In some embodiments, p53 mutation is the load of p53 mutation in the population. In some embodiments, p53 mutation is the frequency of p53 mutation in the population. In some embodiments, p53 mutation is the relative number of p53 mutant cells in the population. In some embodiments, p53 mutation is the rate of p53 mutation in the population. In some embodiments, p53 mutation rate is not altered in the population.

In some embodiments, the method maintains p53 mutation in the population. In some embodiments, the method reduces p53 mutation in the population. In some embodiments, very low prevalence of p53 mutation is maintained. In some embodiments, the very low prevalence is absence of p53 mutation. It will be understood by a skilled artisan that when PSCs are first acquired, whether form an embryo or in the form of iPSCs, the p53 mutation burden will be low and so initiation of a method of the invention will maintain this low mutation level. In contrast if the method is initiated in a population that already has a significant prevalence of p53 mutation, the method of the invention will reduce the p53 mutation levels. In some embodiments, the maintained and/or reduced p53 mutation is as compared to a control. In some embodiments, the control is the same cells and/or cell type that has not undergone a method of the invention. In some embodiments, the control is a predetermined control level of p53 mutation. In some embodiments, the control is cells grown without the activation and/or inhibition of the method of the invention. In some embodiments, the control is cells grown in unsupplemented media appropriate for growth of the cell. A skilled artisan will appreciate that media for the growth of stem cells will be different than media for the growth of differentiated cells. So, while embryonic stem cell media will be supplemented as compared to hematopoietic stem cell media for example, such supplements would still be given to the control cells as this is the standard media for growing this cell type.

In some embodiments, the method further comprises measuring and/or confirming maintained or reduced p53 mutation in the population. In some embodiments, the method further comprises use of the cells in an assay requiring wild type p53 expressing cells. In some embodiments, wild type p53 is non-mutant p53. In some embodiments, the assay is a therapeutic assay. In some embodiments, the assay is a screening assay. In some embodiments, the assay is a culturing assay. In some embodiments, the assay is a differentiation assay. An assay may be any use of the cells that requires functional p53 and/or a lack of mutation in the cells. In some embodiments, the assay is an assay that requires genomic integrity. P53 is a master regulator or mutation in cells and loss of functional p53 can lead to widespread mutation.

In some embodiments, the culturing does not comprise activating and/or inhibiting p53 itself. In some embodiments, the method does not comprise altering p53 or p53 interactors. In some embodiments, the method comprises activating/inhibiting genes, proteins and pathways downstream of p53.

In some embodiments, p53 is TP53. In some embodiments, p53 is tumor protein p53. In some embodiments, the wild-type human p53 gene has the sequence provided in the RefSeq file NM_000546. In some embodiments, the wild-type human p53 gene has the sequence provided in any one of RefSeq file NM_000546, NM_001126112, NM_001126113, NM_001126114, NM_001126115, NM_001126116, NM_001126117, NM_001126118, NM_001276695, NM_001276696, NM_001276697, NM_001276698, NM_001276699, NM_001276760, and NM_001276761. In some embodiments, the wild-type human p53 protein has the sequence provided in the RefSeq file NP 000537. In some embodiments, the wild-type human p53 protein has the sequence provided in any one of RefSeq file NP_000537, NP_001119584, NP_001119585, NP_001119586, NP_001119587, NP_001119588, NP_001119589, NP_001119590, NP_001263624, NP_001263625, NP_001263626, NP_001263627, NP_001263628, NP_001263689, and NP_001263690.

In some embodiments, the culturing does not comprise differentiation of the cells. In some embodiments, the method does not differentiate the cells. In some embodiments, the method does not induce differentiation of the cells. In some embodiments, the method does not increase differentiation. In some embodiments, the increase is as compared to a control. In some embodiments, the differentiation is to mesoderm. In some embodiments, the differentiation is to ectoderm. In some embodiments, the differentiation is to endoderm. In some embodiments, the differentiation is to cardiac cells. In some embodiments, the differentiation is to neuronal cells. In some embodiments, the culturing does not increase apoptosis in the population. In some embodiments, the method does not increase apoptosis in the population. In some embodiments, the increase is as compared to a control. In some embodiments, the culturing does not decrease apoptosis in the population. In some embodiments, the method does not decrease apoptosis in the population. In some embodiments, the decrease is as compared to a control.

In some embodiments, the culturing increases the growth rate of the population. In some embodiments, the method increases the growth rate of the population. In some embodiments, the increased growth rate is of p53 wild-type cells in the population. In some embodiments, p53 mutant cells in the population do not experience a growth rate increase. In some embodiments, the culturing increases the growth rate of p53 wild-type cells in the population. In some embodiments, the method increases the growth rate of p53 wild-type cells in the population. In some embodiments, the increased growth rate is as compared to a control.

In some embodiments, the increase is not less than a 100, 90, 80, 75, 70, 60, 50, 40, 30, 25, 20, 15, 10, 7, 5, 4, 3, 2, 1% increase. Each possibility represents a separate embodiment of the invention. In some embodiments, the increase is not more than a 20, 25, 30, 40, 50, 60, 70, 75, 80, 90, 100, 150, 200, 250, 300, 400, or 500% increase. Each possibility represents a separate embodiment of the invention. In some embodiments, the decrease is not less than a 100, 90, 80, 75, 70, 60, 50, 40, 30, 25, 20, 15, 10, 7, 5, 4, 3, 2, 1% decrease. Each possibility represents a separate embodiment of the invention. In some embodiments, the decrease is not more than a 100, 90, 80, 75, 70, 60, 50, 40, 30, 25, 20, 15, 10, 7, 5, 4, 3, 2, 1% decrease. Each possibility represents a separate embodiment of the invention.

In some embodiments, the method comprises inhibiting at least one of Bax, Noxa, PERP and PAG608. In some embodiments, the method comprises at least one of activating mTOR signaling and inhibiting at least one of Bax, Noxa, PERP and PAG608. In some embodiments, the method comprises at least 1, 2, 3, 4 or 5 of activating mTOR signaling and inhibiting at least one of Bax, Noxa, PERP and PAG608. Each possibility represents a separate embodiment of the invention.

In some embodiments, Bax is BCL2 associated X, apoptosis regulator. In some embodiments, the Bax gene has the sequence provided in the RefSeq file NM_138761. In some embodiments, the human Bax gene has the sequence provided in any one of RefSeq file NM_138761, NM_004324, NM_138763, NM_138764, NM_001291428, NM_001291429, NM_001291430, NM_001291431, NM_027882 and XM 017027077. In some embodiments, the wild-type human Bax protein has the sequence provided in the RefSeq file NP_620116. In some embodiments, the wild-type human Bax protein has the sequence provided in any one of RefSeq file NP_004315, NP_620118, NP_620119, NP_001278357, NP_001278358, NP_001278359, NP_001278360, XP_016882566.

In some embodiments, Noxa is PMAIP1. In some embodiments, the Noxa gene has the sequence provided in the RefSeq file NM_021127. In some embodiments, the human Noxa protein has the sequence provided in the RefSeq file NP_066950.

In some embodiments, the PERP gene has the sequence provided in the RefSeq file NM_022121. In some embodiments, the PERP gene has the sequence provided in any one of RefSeq file NM_022121 and XM_024446520. In some embodiments, the human PERP protein has the sequence provided in the RefSeq file NP_071404. In some embodiments, the human PERP protein has the sequence provided in any one of RefSeq file NP_071404 and XP_024302288.

In some embodiments, PAG608 is zinc finger martin-type 3. In some embodiments, PAG608 is ZMAT3. In some embodiments, the PAG608 gene has the sequence provided in the RefSeq file NM_022470. In some embodiments, the PAG608 gene has the sequence provided in any one of RefSeq file NM_022470, NM_152240, XM_005247706 and XM_011513073. In some embodiments, the human PAG608 protein has the sequence provided in the RefSeq file NP_071915. In some embodiments, the human PAG608 protein has the sequence provided in any one of RefSeq file NP_071915, NP_689426, XP_005247763 and XP_011511375.

In some embodiments, inhibiting comprises inhibiting expression. In some embodiments, expression is mRNA expression. In some embodiments, expression is protein expression. In some embodiments, inhibiting is inhibiting function. In some embodiments, inhibiting is inhibiting expression, function or both. In some embodiments, inhibiting expression comprises inhibiting translation. In some embodiments, inhibiting expression comprises inhibiting transcription. In some embodiments, inhibiting expression comprises mRNA degradation.

In some embodiments, inhibiting comprises contacting the population of cells with an inhibitor. In some embodiments, activating comprises contacting the population of cells with an activator. In some embodiments, the contacting comprises supplementing media comprises the PSCs with an activator and/or inhibitor. In some embodiments, the contacting is by transfection. In some embodiments, the contacting is by diffusion. In some embodiments, the activator/inhibitor is targeted to the cells. In some embodiments, the contacting is administering an activator, inhibitor or both.

As used herein, an “activator” refers to a molecule that induces, activates or enhances signaling in a signaling pathway. As used herein, an “inhibitor” is a molecule that inhibits, stops or reduces functionality of a target. Assays for testing activator/inhibitor function are well known in the art. mTOR activation can be measured by readout such as Akt phosphorylation, 4E-BP1 phosphorylation, protein synthesis levels, and S6K1 phosphorylation. Methods of monitoring mTOR activity can be found in Ikenoue et al., Chapter 11 Monitoring Mammalian Target of Rapamycin (mTOR) Activity, Methods in Enzymology, Volume 452, 2009, Pages 165-180, herein incorporated in its entirety by reference. Inhibitors of BAX, Noxa, PERP, and PAG608 can be assessed by measuring caspase activity, cytochrome C levels, mRNA and protein levels of the 4 targets, cellular respiration and mitochondrial function.

In some embodiments, the method of the invention further comprises selecting an mTOR activator. In some embodiments, the method of the invention further comprises selecting a BAX inhibitor. In some embodiments, the method of the invention further comprises selecting a Noxa inhibitor. In some embodiments, the method of the invention further comprises selecting a PERP inhibitor. In some embodiments, the method of the invention further comprises selecting a PAG608 inhibitor. In some embodiments, the method of the invention further comprises confirming at least one of mTOR activation, BAX inhibition, Noxa inhibition, PERP inhibition, and PAG608 inhibition.

In some embodiments, activating comprises an increase of at least 20, 25, 30, 40, 50, 60, 70, 75, 80, 90, 95, 99, 100, 150, 200, 250, 300, 350, 400, 450 or 500%. Each possibility represents a separate embodiment of the invention. In some embodiments, inhibiting comprises a decrease of at least 20, 25, 30, 40, 50, 60, 70, 75, 80, 90, 95, 97, 99, or 100%. Each possibility represents a separate embodiment of the invention. In some embodiments, an activator activates by at least an increase of 20, 25, 30, 40, 50, 60, 70, 75, 80, 90, 95, 99, 100, 150, 200, 250, 300, 350, 400, 450 or 500%. Each possibility represents a separate embodiment of the invention. In some embodiments, an inhibitor inhibits by at least 20, 25, 30, 40, 50, 60, 70, 75, 80, 90, 95, 97, 99, or 100%. Each possibility represents a separate embodiment of the invention.

In some embodiments, the inhibitor/activator is a chemical inhibitor/activator. In some embodiments, the inhibitor/activator is a small molecule. In some embodiments, the inhibitor/activator is an RNA. In some embodiments, the RNA is selected from a miRNA, an siRNA, an shRNA, a lncRNA and an anti-sense oligonucleotide. In some embodiments, the inhibitor inhibits mRNA stability. In some embodiments, the inhibitor inhibits mRNA translation. In some embodiments, the inhibitor inhibits protein stability. In some embodiments, the inhibitor inhibits protein function. In some embodiments, the inhibitor inhibits at least one of mRNA stability, mRNA translation, protein stability and protein function. In some embodiments, the activator/inhibitor is a direct activator/inhibitor. In some embodiments, the activator/inhibitor is an indirect activator/inhibitor. As used herein, a “direct activator/inhibitor” is a molecule that binds directly to gene/protein of interest and not to an upstream or downstream target. For example, a direct inhibitor of Bax binds to the Bax gene, a Bax mRNA or the Bax protein (BIP-V5 for non-limiting example). In some embodiments, the activator/inhibitor is a specific activator/inhibitor. As used herein, the term “specific activator/inhibitor” is a molecule that specially inhibits a particular protein and/or pathway. For example, a Bax specific inhibitor would only inhibit Bax and not other genes/proteins substantially. In some embodiments, specific activation/inhibition comprises not more than a 1, 2, 3, 4, 5, 7, 10, 15, 20 or 25% activation or inhibitor of an off-target gene, protein or pathway. Each possibility represents a separate embodiment of the invention.

In some embodiments, activating mTOR signaling comprises activating the mTOR pathway. In some embodiments, activation mTOR comprises activation of mTORC1. In some embodiments, activation of mTOR comprises activation of mTORC2. In some embodiments, activation of mTOR comprises activation of mTORC1, mTORC2 or both. In some embodiments, activating mTOR signaling comprises activating PDK1. In some embodiments, activating mTOR signaling comprises activating AKT1. In some embodiments, a PDK1 activator is PS48. In some embodiments, an AKT1 activator is SC79. In some embodiments, activating mTOR signaling comprises inhibiting an inhibitory protein of the mTOR pathway. In some embodiments, the inhibitory protein is selected from PTEN TSC1 and TSC2. In some embodiments, activating mTOR signaling comprises inhibiting expression, function or both of at least one of PTEN, TSC1 and TSC2. In some embodiments, a PTEN inhibitor is VO-OHpic. In some embodiments, an activator of mTOR signaling is insulin like growth factor 1 (IGF1). Examples of activators of mTOR signaling are well known in the art and include, but are not limited to: MHY1485, 3-benzyl-5-((2-nitrophenoxy) methyl)-dihydrofuran-2(3H)-one (3BDO), TSC1 knockdown agents (miRNA, siRNA, shRNA for example), PDK1 activators, AKT1 activators, TSC2 knockdown agents, PTEN knockdown agents and IGF1.

Examples of Bax inhibitors include, but are not limited to, Bax inhibiting peptide V5 (BIP-V5), Bax knockdown agents, Bax inhibitor 1, BJ-1, BJ-1-BP, MSN-50, MSC-125 and BAIs. Examples of Noxa inhibitors include, Noxa knockdown agents. Examples of PERP inhibitors include, but are not limited to, PERP knockdown agents, and PERP neutralizing antibodies. Examples of PAG608 inhibitors include, PAG608 knockdown agents.

In some embodiments, the methods of the invention are performed ex vivo. In some embodiments, the methods of the invention are performed in vitro. In some embodiments, the methods of the invention are performed in culture. In some embodiments, the methods of the invention are performed in a subject. In some embodiments, the subject is a non-human. In some embodiments the subject is a human.

By another aspect, there is provided a method of inducing apoptosis in an ESC comprising inhibiting mTOR signaling in the ESC thereby inducing apoptosis.

In some embodiments, the ESC is in a mixed population of ESCs and non-ESC cells and the apoptosis is specific to ESCs. In some embodiments, the other cells are cells differentiated from ESCs.

By another aspect, there is provided a method of differentiating ESC, the method comprising providing a culture of undifferentiated ESCs, differentiating at least a portion of the ESCs to a non-pluripotent cell type and inhibiting mTOR signaling in the culture, thereby differentiation ESC.

In some embodiments, the inhibiting mTOR signaling is performed for a time sufficient to induce apoptosis in the undifferentiated ESCs. In some embodiments, the inhibiting mTOR signaling is performed for a time that does not induce apoptosis in the differentiated cells. In some embodiments, the method is a method of producing a pure population of differentiated ESCs. Methods of ESC differentiation are well known in the art, and any method that results in at least a portion of the ESCs differentiating to a non-pluripotent cell type may be employed.

In some embodiments, inhibiting mTOR signaling comprises contacting the cells with an mTOR inhibitor. In some embodiments, contacting the cells with an mTOR inhibitor comprises administering an mTOR inhibitor. In some embodiments, contacting the cells with an mTOR inhibitor comprises adding an mTOR inhibitor to the culture. Examples of mTOR inhibitors include, but are not limited to, rapamycin, carvedilol, testosterone propionate, hydroxyprogesterone, sirolimus, temsirolimus, everolimus, ridaforolimus, and other rapalogs. In some embodiments, the inhibitor is a first-generation inhibitor. In some embodiments the inhibitor is a second-generation inhibitor.

By another aspect, there is provided cell culture media comprising a Bax inhibitor.

By another aspect, there is provided cell culture media comprising a Noxa inhibitor.

By another aspect, there is provided cell culture media comprising a PERP inhibitor.

By another aspect, there is provided cell culture media comprising a PAG608 inhibitor.

By another aspect, there is provided cell culture media comprising an mTOR activator.

In some embodiments, the cell culture media comprises at least one of:
a. an mTOR activator;
b. a Bax inhibitor;
c. a Noxa inhibitor;
d. a PERP inhibitor; and
e. a PAG608 inhibitor.

In some embodiments, the cell culture media is stem cell media. In some embodiments, the media is human cell culture media. In some embodiments, the stem cell media is PSC media. In some embodiments, the PSC media is ESC media. In some embodiments, the PSC media is iPSC media. In some embodiments, the PSC media is selected from ESC media and iPSC media. In some embodiments, the cell culture media is media supplemented with at least one of: an mTOR activator, a Bax inhibitor, a Noxa inhibitor, a PERP inhibitor and a PAG608 inhibitor.

Stem cell media, and in particular PSC media, are well known in the art and are available commercially. Such medias are available from ReproCell, Thermo Fisher, STEMCELL Technologies, and Biological Industries to name but a few. Examples of PSC media include, but are not limited to NutriStem hPSC medium, Primate ES cell medium, StemFit Basic, CTS KnockOut Serum Replacement XenoFree Medium, StemFlex Medium and Essential 8 medium to name but a few.

In some embodiments, the inhibitor is a direct inhibitor of Bax. In some embodiments, the inhibitor is a direct inhibitor of Noxa. In some embodiments, the inhibitor is a direct inhibitor of PERP. In some embodiments, the inhibitor is a direct inhibitor of PAG608. In some embodiments, the inhibitor does not directly inhibit any protein or gene other than at least one of Bax, Noxa, PERP and Pag608.

By another aspect, there is provided a method of producing an induced pluripotent stem cell (iPSC), the method comprising expressing in a non-pluripotent cell MYB proto-oncogene like 2 (MYBL2).

In some embodiments, the non-pluripotent cell is a somatic cell. In some embodiments, the cell is a fibroblast. In some embodiments, the non-pluripotent cell is an adult cell selected from a fibroblast, a keratinocyte, an epithelial cell, a hematopoietic cell and a mesenchymal cell.

In some embodiments, the method further comprises expressing in the non-pluripotent stem cell at least one gene selected from Sall4, OCT4, PRDM14, NANOG, FOXB1 and MYCN. In some embodiments, the method comprises expressing in the non-pluripotent stem cell MYBL2, Sall4, OCT4, PRDM14, NANOG, FOXB1 and MYCN.

In some embodiments, the method further comprises expressing in said somatic non-pluripotent cell at least one of SRY-box 2 (SOX2), Kruppel-like factor 4 (KLF4), c-MYC, and 1-MYC.

By another aspect, there is provided a kit comprising at least one expressing vector capable of inducing expression in a mammalian cell of at least four genes selected from MYBL2, SALL4, OCT4, PRDM14, NANOG, FOXB1 and MYCN. In some embodiments, the kit comprises an expression vector capable of inducing expression of MYBL2 in a mammalian cell.

In some embodiments, the expression vector comprise a mammalian promotor operatively linked to a coding region encoding at least one gene.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

### Cell Lines, Vectors and Reagents

The following cell lines were used in this study—haploid hESCs: h-pES10 cell line; REX1-EGFP cells: hESCs carrying the eGFP gene under the REX1 promoter; 293T cells: obtained from R. Weinberg (Whitehead Institute); BJ human fibroblasts: purchased from Clontech; KBM7 cells: purchased from Horizon Discovery. The activity-optimized Human CRISPR Pooled Library (cat. no. 1000000067), pCMV-VSV-G (cat. no. 8454), psPAX2 (cat. no. 12260) and LentiCRISPR v2 (cat. no.: 52961) were purchased from Addgene. IGF1 was obtained from PeproTech. Rapamycin was obtained from Cayman Chemical Company. Torin 1 was obtained from Cell Signaling.

### Cell Culture

Haploid hESCs were cultured at 37° C. and 5% CO2 on feeder layer growth-arrested mouse embryonic fibroblasts (MEFs) in standard hESC growth medium, composed of knockout Dulbecco's modified. Eagle's medium (DMEM) supplemented with 15% knockout serum replacement (KSR, Thermo Fisher Scientific), 2 mM L-glutamine, 0.1 mM nonessential amino acids, 50 units ml⁻¹ penicillin, 50 μg ml⁻¹ streptomycin, 0.1 mM β-mercaptoethanol and 8 ng ml⁻¹ basic fibroblast growth factor (bFGF). Cells were passaged by a quick trypsinization using trypsin-EDTA (Biological Industries) and plated in the presence of 10 μM ROCK inhibitor Y-27632 (Stemgent) for 1 day after splitting. BJ fibroblasts, feeder layer MEFs and 293T cells were cultured in DMEM supplemented with 10% fetal bovine serum (Invitrogen), 2 mM L-glutamine, 50 units ml⁻¹ penicillin and 50 μg ml⁻ streptomycin. REX1-EGFP WA09, CSES9 and CSES15 hESC lines were cultured in feeder-free conditions on matrigel-coated plates (Corning) in mTeSR1 (STEMCELL Technologies). KBM7 cells were cultured in Iscove's modified Dulbecco's medium (IMDM) supplemented with 10% fetal bovine serum (Invitrogen), 2 mM L-glutamine, 50 units ml⁻¹ penicillin and 50 μg ml⁻¹ streptomycin. Cell lines were free of mycoplasma.

Enrichment of Haploid hESCs

Haploid hESCs were enriched as described in Sagi, I., et al. Identification and propagation of haploid human pluripotent stem cells. Nat. Protoc. 11, 2274-2286 (2016), herein incorporated by reference in its entirety. Cells were washed with phosphate buffered saline (PBS), trypsinized using TrypLE Select (Thermo Fisher Scientific) and stained with 10 μg ml⁻¹ Hoechst 33342 (Sigma Aldrich) in hESC growth medium at 37° C. for 30 min. Cells were then centrifuged and resuspended in PBS containing 10% KSR and 10 μM ROCK inhibitor Y-27632, filtered through a 70 μm cell strainer (Corning) and sorted by a 405 nm laser in BD FACSAria III (BD Biosciences). On plating the sorted cells, 10 μM ROCK inhibitor Y-27632 was added to the medium for one day.

Library Plasmid Amplification, Virus Production and Transduction of Haploid hESCs

sgRNA library cloned into Cas9-containing lentiCRISPR v1 plasmids was transformed into Endura electrocompetent cells (Lucigen). Transformed cells were plated on ampicillin-containing agar plates (Sigma) and used for plasmid isolation. To maintain the diversity of the sgRNA library, more than 100-fold coverage of the size of the sgRNA library was achieved in the number of transformed colonies (>18 million colonies).

To produce virus library for 181,131 sgRNAs, 293T cells in forty 15 cm culture plates with around 70-80% confluency were transfected with sgRNA-containing lentiCRTSPR v1, pCMV-VSV-G and psPAX2 plasmids at a ratio of 13.16.6:10 (30 total per plate), respectively, in the presence of polyethylenimine ‘Max’ (Polysciences) Transfection medium was exchanged with 0.5% BSA-containing 293T growth medium after 16 h, and lentiviral particle-containing culture supernatant was harvested 60-65 h after transfection. Culture supernatant was spun down at 3,000 r.p.m. for 10 min at 4° C. and then filtered through 0.45 μm cellulose acetate filters (Millipore). Filtered supernatant was centrifuged in a swing bucket rotor (Beckman Coulter) at 24,000 r.p.m. for 2 h at 4° C. The pellet was very briefly dried, then reconstituted in cold hESC growth medium (<1 ml) and frozen in aliquots at −70° C. Virus titres were measured. A total of 378 million haploid-enriched hESCs were transduced with the virus library at a multiplicity of infection (MOI) of 0.3, resulting in infection of 30% of the cells and hence leading to a 700-fold coverage of the sgRNA library size. A MOI of 0.3 ensures a high enrichment in the proportion of cells that are infected with only one viral particle and therefore carry a single mutation. Haploid hESCs can be sorted to purity from a mixed population of haploid and diploid cells. We transduced hESCs a week after haploid cell enrichment, when about 90% of the cells are still haploid. After the introduction of sgRNAs, diploidization would lead to the generation of homozygous mutations through endoduplication of mutant cells, and hence it is not expected to affect the effectiveness of loss-of-function mutations.

For transduction, haploid hESCs were trypsinized with trypsin-EDTA, centrifuged and resuspended in hESC growth medium supplemented with 10 μM ROCK inhibitor Y-27632 and 8 μg ml⁻¹ polybrene (Sigma). The viruses were then added to the cell suspension. Transduced haploid hESCs were densely plated on feeder layer MEFs overnight (3 million cells in 1.5 ml hESC medium for one well of a six-well plate). At 24 h after transduction, cells were passaged on a feeder layer of DR3 MEFs at a ratio of 1:3 in the presence of 5 μM ROCK inhibitor Y-27632. During this passaging, 35 million cells were harvested for DNA extraction and sgRNA analysis for the ‘Day 1’ time point after infection. At 12 h after this passaging, the medium of the cells was replaced with puromycin-containing medium (0.3 mg ml Sigma). Cells were kept under puromycin selection for 7 days and then passaged again. At 9 days after initial transduction, 55 million cells (300-fold the size of the sgRNA library) were collected and mixed for DNA extraction and sgRNA analysis for the ‘Day 9’ time point after infection. Transduced haploid hESCs were passaged every 4-5 days while maintaining at least 400-fold representation of the sgRNA library. Fifty-five million cells were also collected for each time point ‘Day 23’ and ‘Day 30’ after infection.

DNA Extraction, PCR Amplification of sgRNAs and High-Throughput DNA Sequencing

Genomic DNA was extracted with a Blood & Cell Culture DNA Midi Kit (QIAGEN) according to the manufacturer's instructions. The region containing the sgRNA integration was amplified with the following primers, which also contain overhang sequences compatible for Nextera DNA library preparations (Illumina): 5′-TCGTCGGCAGCGTCAGATGTGTATAAGAGACAGGGCTTTATATATCTTGTGG AAAGGACG-3′ (SEQ ID NO: 1) (forward) and 5′-GTCTCGTGGGCTCGGAGATGTGTATAAGAGACAGACGGACTAGCCTTATTTT AACTTGC-3′ (SEQ ID NO: 2) (reverse).

The total genomic DNA for each time point was divided into 50 μl PCR reactions with 4 μg DNA input. The PCR settings have been described in Wang, T., et al. Viral packaging and cell culture for CRISPR-based screens. Cold Spring Harb. Protoc. doi.org/gcw7js (2016) herein incorporated by reference in its entirety. After purification of the 160-base-pair (bp) product, a second PCR reaction was performed using Nextera adapter primers to generate a Nextera DNA library according to the manufacturer's instructions (Illumina). DNA libraries containing sgRNA constructs from two replicate experiments were sequenced using NextSeq 500 (Illumina).

### Data Analysis

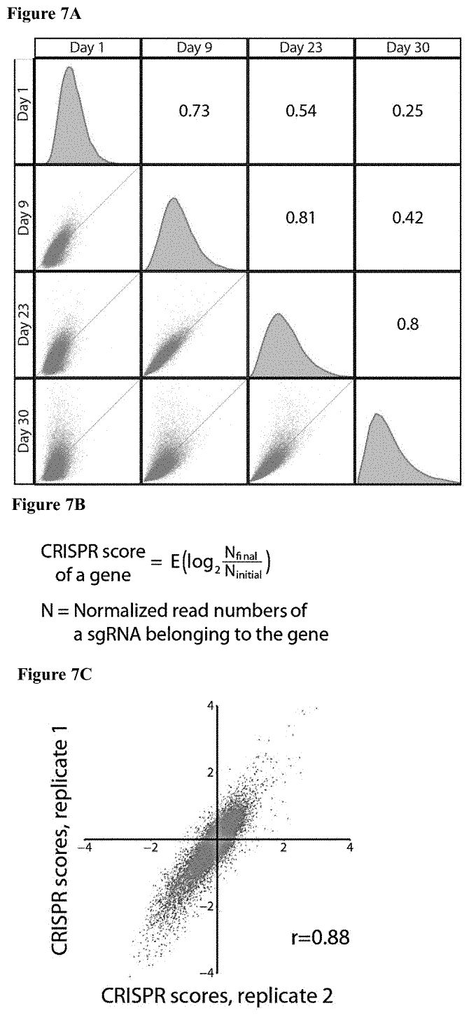

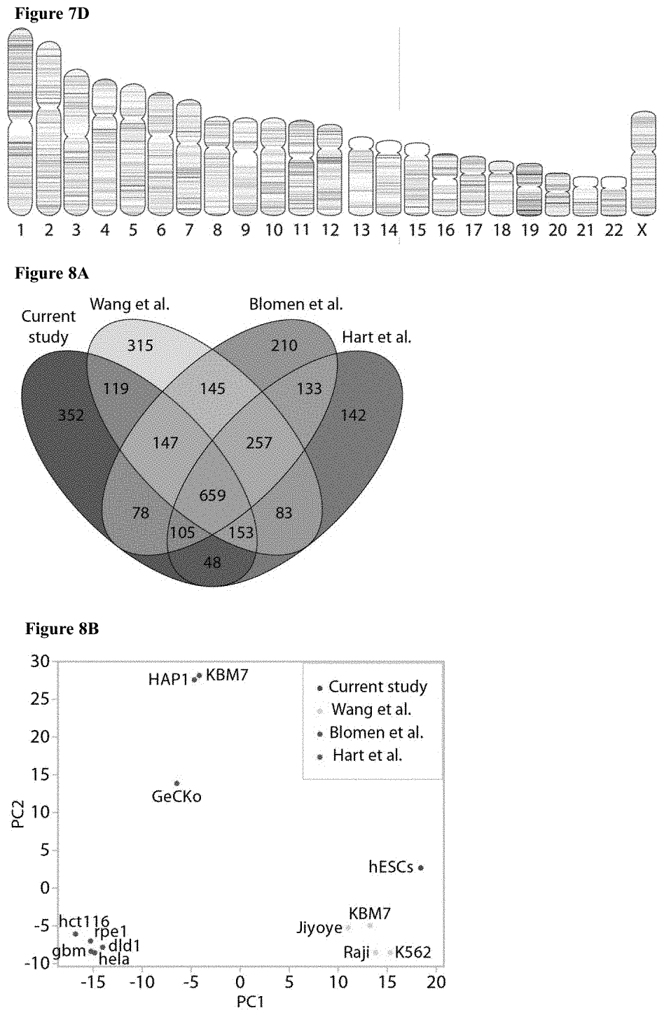

The numbers of reads obtained from sequencing were 80.5 million and 68.6 million reads for day 1, 71.5 million reads for day 9, 84.1 million and 70.4 million reads for day 23, and 43.2 million reads for day 30 after introduction of the gRNAs. Mapping was performed by aligning the sgRNA sequences to the reads (treating the reads as a reference genome) using the bowtie2 program, and analysing only complete 20-base matches. The minimal number of reads mapped to any of the sgRNAs under any of the conditions was 4. The count table was then normalized relative to the total number of reads in each of the conditions, and replicates were averaged. CRISPR scores are the average of the log₂ ratios of the abundance of all sgRNAs for each gene between final (day 23) and initial (day 1) populations (FIG. 7B). Statistical significance was determined by the Kolmogorov-Smirnov test for two samples, using ks_2samp from python's scipy.stats module. In doing so, each population of sgRNAs belonging to a gene was compared to the general distribution of sgRNAs from the same condition. The Benjamini-Hochberg FDR correction was accomplished with the multipletests feature from python's statsmodels.sandbox.stats.multicomp module.

### Comparison of Genetic Screens in Haploid Versus Diploid Cells

Induction of loss of function, using the CRISPR-Cas9 methodology, may be more efficient in haploid than diploid cells for several reasons. The Cas9 endonuclease guided by the sgRNA creates a double-strand break that can lead to nucleotide insertions or deletions (indels) due to the non-homologous-end-joining mechanism. In the majority of cases, these indels will be one or two nucleotides, creating a frameshift and a loss of function in the allele. However, in some cases, allelic loss of function does not occur, mainly due to indels in multiples of three nucleotides preserving the reading frame, but also due to implementation of the homologous-recombination repair mechanism or the lack of a double-strand-break reaction. Let L be the allelic loss-of-function rate, then, for a given sgRNA, the probability of successfully targeting both alleles in a diploid cell is L\hat{ }2, while in a haploid cell this rate is L. Hence, for a specific sgRNA, we would expect the loss-of-function rate to L/L\hat{ }2=1/L times higher in haploids than in diploids. Thus, if indels in the multiples of three nucleotides occur in a third of the mutations, frameshift mutations will occur 50% more frequently in a haploid allele than in diploid alleles.

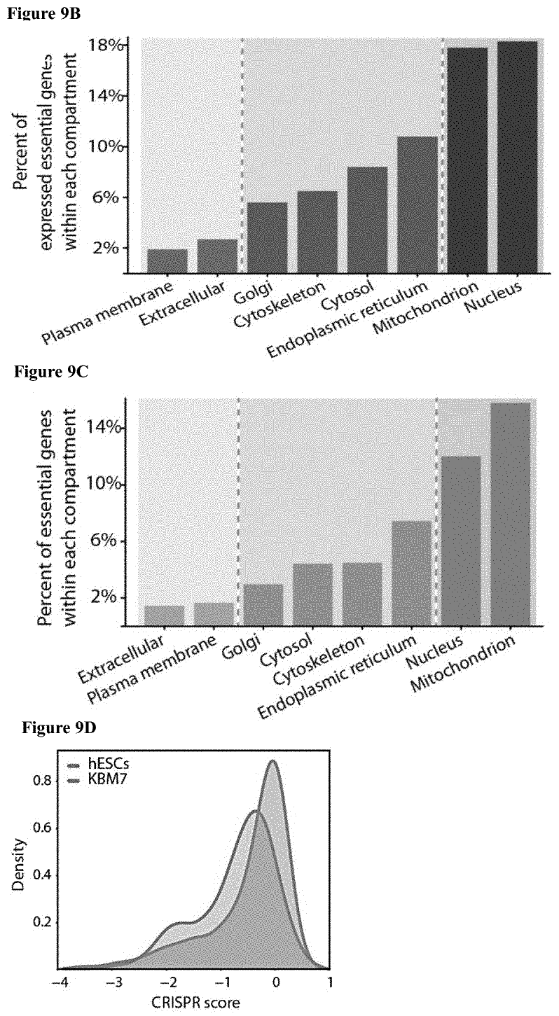

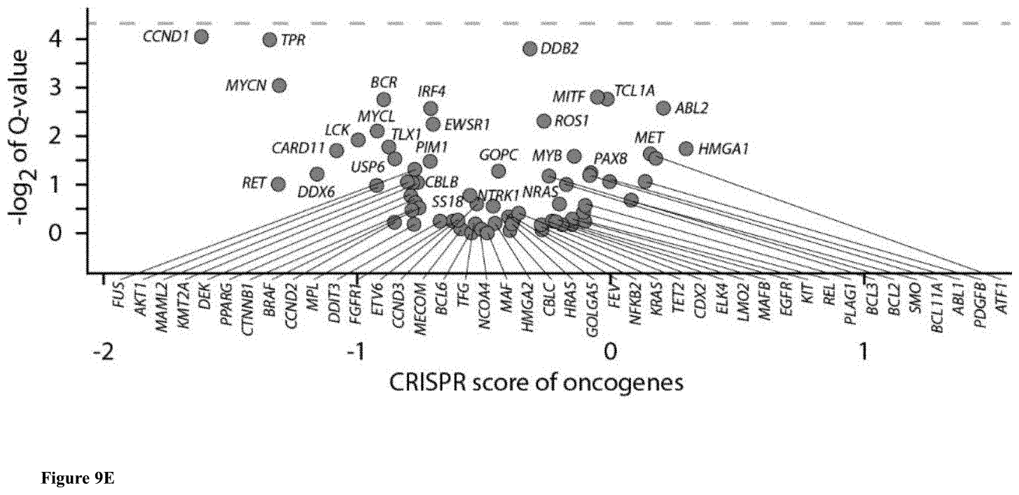

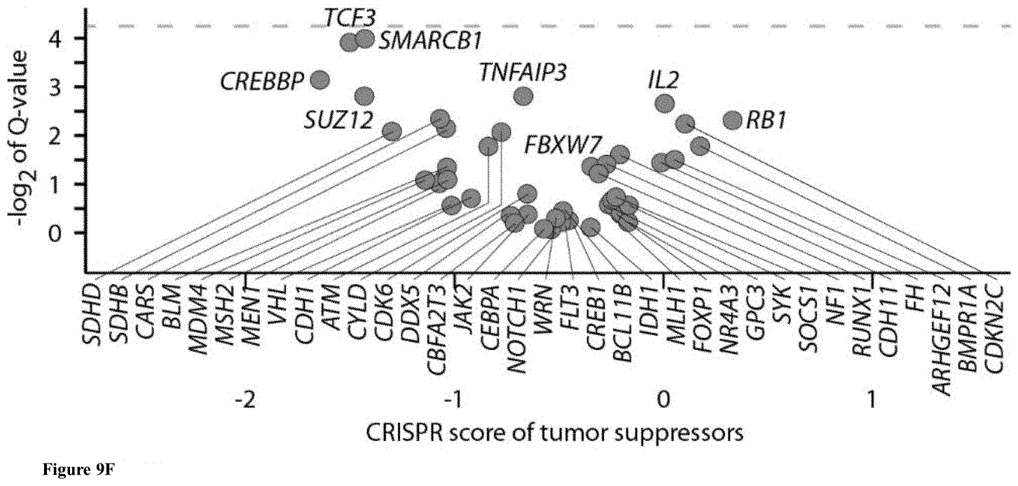

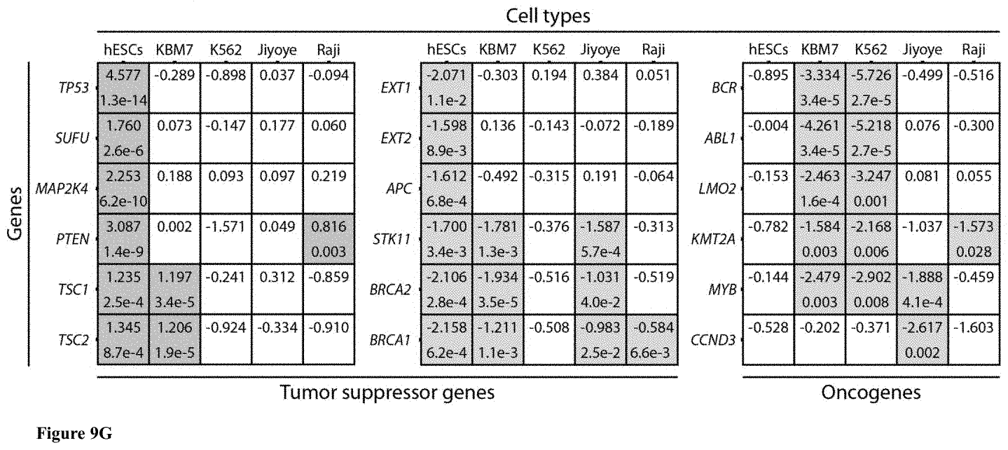

The assumption that complete loss-of-function alleles are more prevalent in haploid than in diploid chromosomes is also supported by data from the near-haploid KBM7 cells, where chromosome 8 is the only full diploid chromosome. The first percentile CRISPR scores in diploid chromosome 8 and the other haploid autosomes in KBM7 cells had been determined. As shown in FIG. 9C, chromosome 8 shows significantly different values than the other chromosomes, suggesting that it is more efficient to achieve loss-of-function mutation in haploid chromosomes. Analysing values for chromosome 8 in three all-diploid cell lines (K562, Jiyoye and Raji) or in the all-haploid cell line (hESCs), showed that chromosome 8 is not different from the other autosomes (FIGS. 9C-D). The data support the notion that although genetic screens using CRISPR-Cas9 technology are fairly efficient in diploid cells, the use of haploid cells provides a further advantage in generating complete loss-of-function frameshift mutations.

### Analysis of Cellular Compartments

Localization of proteins into cellular compartments was retrieved from the Subcellular Localization Database website (compartments.jensenlab.org/About), where each gene is given a number of compartments with matching confidence level scores. For each of the genes we defined the maximal confidence score, and assigned it with the compartments. We then analysed the genes that were associated with a single compartment. Among 18,099 genes in our study, 17,242 (95%) were assigned to compartments and 10,932 (63%) were associated with a single compartment. Statistical significance for the enrichment of nuclear and mitochondrial compartments among essential genes was assessed by the hypergeometric test.

### Analysis of AR Disorders and Cancer-Related Genes

To analyse the involvement of genes responsible for genetic disorders in the growth of hESCs we utilized the database of the Online Mendelian Inheritance in Man (OMIM) (omim.org/), which lists diseases associated with genes, their pattern of inheritance, and their clinical synopsis. Of the annotated genes in OMIM, gRNAs for 3,592 genes appear in the library, and 2,099 of them have an AR inheritance. Of these AR inheritance genes, 766 also had a growth retardation-related phenotype. The FA genes are one example of such genes: FANCA (FA-A), FANCB (FA-B), FANCC (FA-C), BRCA2 (FA-D1), FANCD2 (FA-D2), FANCE (FA-E), FANCF (FA-F), FANCG (FA-G), FANCI (FA-I), BRIP1 (FA-J), FANCL (FA-L), FANCM (FA-M), PALB2 (FA-N), RAD51C (FA-O) and SLX4 (FA-P).

The list of canonical oncogenes and tumour suppressor genes were retrieved from Wang, T. et al. Identification and characterization of essential genes in the human genome. Science 350, 1096-1101 (2015) herein incorporated by reference in its entirety.

Analysis of hESC-Enriched Genes

Defining genes with enriched expression in hESCs was performed by comparing expression data from 26 tissues to that of 10 hESC lines from four different studies (SRR2038465, SRR2038466, SRR2038467, SRR2038469, SRR2038474, SRR2038475, SRR2038476, SRR2038477, SRR2453342, SRR2453346, SRR2453356, SRR2453360, SRR2453365, SRR2453368, SRR2453370, SRR3382655, SRR3575052). The tissue expression data was retrieved from the GTEx Portal version V6p (GTEx_Analysis_v6p_RNA-seq_RNA-SeQCv 1.1.8_gene_reads.gct), normalized together with the data from hESC lines, and averaged over similar tissues. Overall, the average expression data represented the following number of samples of each category: 17 hESCs, 285 transformed fibroblasts, 119 transformed lymphocytes, 815 brain, 689 oesophagus, 609 skin, 579 adipose, 431 skeletal muscle, 414 heart, 394 whole blood, 347 colon, 324 thyroid, 321 lung, 605 nerve, 215 breast, 194 stomach, 172 pancreas, 146 adrenal gland, 120 liver, 107 prostate, 105 spleen, 104 pituitary, 89 small intestine, 84 uterus, 58 salivary gland, 33 kidney, 12 bladder. For a gene to be considered as expressed in hESC, its averaged FPKM level has to surpass 1. Genes that were considered enriched in hESC are at least 10 times more expressed in hESC than any other tissue and at least 3 times more expressed relative to transformed cell lines.

Comparison between hESC and EB expression was performed using expression data from hESC and EB samples from the same haploid cell line (SRR2131924, SRR2131925, SRR2131926, SRR2131927, SRR2131929, SRR2131937).

siRNA Knockdown, Cell Viability and Growth Curve Assays

All genes were targeted with commercial pooled siRNAs to increase the specificity of the knockdown. esiRNAs for SALL4, DSCC1 and SEPHS1 and the control esiRNA for Renilla luciferase were obtained from Sigma (cat. nos. EHU037061, EHU021301, EHU107861 and EHURLUC, respectively). siRNA for VRTN was obtained from GE Healthcare Dharmacon (cat. no. L-021159-02-0005). Briefly, 30-50 nM siRNA was mixed with 0.14 μl of DharmaFECT 1 transfection reagent (GE Healthcare Dharmacon) in Opti-MEM I reduced serum medium (Thermo Fisher Scientific) for 30 min. The transfection mix was added on a matrigel-coated well of a 96-well plate and 3,000 hESCs were plated on the transfection mix in the presence of mTeSR 1 medium. The cells were subsequently grown for 3-4 days and the mTeSR 1 medium was replaced every 24 h. Cell viability was assessed by a CellTiter-Glo luminescent cell viability assay according to the manufacturer's instructions (Promega). Luminescence reads for the target genes were normalized to control siRNA conditions, and the replicate experiments were averaged. For growth curves of IGF1-treated and control hESCs, cells were plated in matrigel-coated wells with equal numbers and their density was measured for four consecutive days with CellTiter-Glo luminescent cell viability assay (Promega). The density measured one day after plating was considered Day 0, after which cells were switched to MEF-conditioned medium containing 100 ng ml⁻¹ IGF1. Every day was normalized to Day 0 and replicate experiments were averaged. For rapamycin and Torin 1 treatment experiments, cells were cultured in mTeSR 1 medium and the drug-containing medium was replaced every 24 h.

Generation of Stable sgRNA Cell Lines

sgRNA sequences used for cloning into lentiCRISPR v2 lentiviral vector were as follows: 5′-GCGCTCTTCAGATCCACGAG-3′ (SEQ ID NO: 3) for SALL4, 5′-GCAGAGTGTTCCTGAAGGAA-3′ (SEQ ID NO: 4) for DSCC1, 5′-CACGTGGTAAACAGATCAGA-3′ (SEQ ID NO: 5) for SEPHS1, 5′-GCACTGGCGGTGTCAAGCCC-3′ (SEQ ID NO: 6) for VRTN, 5′-ACAGCCACACACTACATCAG-3′ (SEQ ID NO: 7) for PIK3CA and 5′-GCTGGCCAGCACAGACGCTG-3′ (SEQ ID NO: 8) for PDIA4A. Viruses containing these constructs were packaged as described above. For the control lines, lentiCRISPR v2 vector without any sgRNA was used. REX1-EGFP hESC and KBM7 cells were transduced with the viral supernatant and the transduced cells were selected with puromycin (0.3 mg ml⁻¹ for hESCs, 2-4 mg ml⁻¹ for KBM7) one day after infection. Three days after infection the cells were plated on 96-well plates for the cell viability assay, and collected for analysis of the wild-type transcript levels of targeted genes. Cell viability was assessed by CellTiter-Glo luminescent cell viability assay (Promega) four days after infection.

### RNA Isolation, RNA Sequencing and Quantitative Real-Time PCR

For high-throughput RNA sequencing experiments with siRNA knockdown, cells were collected 48 h after transfection of siRNAs for SALL4 and VRTN and 72 h for DSCC1 and SEPHS1. Total RNA was isolated from three independent biological replicates with RNeasy Mini or Micro Kit (QIAGEN) and the mRNA fraction of total RNA was enriched by pulldown of poly(A)-RNA. RNA sequencing libraries were generated using SENSE Total RNA-Seq Library Prep Kit (LEXOGEN) according to the manufacturer's protocol and sequenced using Illumina NextSeq 500 with 85 bp single-end reads. For rapamycin experiments, RNA sequencing libraries were generated using the Illumina TruSeq RNA prep kit v2 according to the manufacturer's protocol and sequenced using Illumina NextSeq 500 with 84 bp single-end reads. For transcriptome analysis, reads were mapped to the GRCh38 reference genome using STAR. Statistical significance was then determined by two-tailed unpaired Student's t-test, and GO enrichment analysis was done by DAVID. Statistical significance was determined using the Benjamini correction.

For qRT-PCR experiments, total RNA was reverse-transcribed into first-strand complementary DNA (cDNA) (Quantabio). The qRT-PCR reaction consisted of initial incubation at 50° C. for 2 min and denaturation at 95° C. for 10 min. The cycling parameters were as follows: 95° C. for 15 s and 60° C. for 30 s. After 40 cycles, the reactions underwent a final dissociation cycle as follows: 95° C. for 15 s, 60° C. for 1 min, 95° C. for 15 s and 60° C. for 15 s. Expression of each gene was normalized to GAPDH expression. The primer sequences used in qRT-PCR reactions to test the siRNA knockdowns were as follows: 5′-TTGAGGGGAGATGGGTACTG-3′ (SEQ ID NO: 9) and 5-AATAAGATGGGGACAGGGTTG-3′ (SEQ ID NO: 10) for SALL4, 5′-TTAGCCTTCCACCCAAACTG-3′ (SEQ ID NO: 11) and 5-TCCCAAAGCGCATGTCTAC-3′ (SEQ ID NO: 12) for DSCC1, 5′-AGGCATTACCCGTAGTCGTG-3′ (SEQ ID NO: 13) and 5-TCCAGAAAACCATTCAAGACG-3′ (SEQ ID NO: 14) for SEPHS1, 5′-TGAGGCACTGGAGATCACTG-3′ (SEQ ID NO: 15) and 5-GGGCCATAATCTGCAAACAG-3′ (SEQ ID NO: 16) for VRTN, 5-AGCCACATCGCTCAGACACC-3′ (SEQ ID NO: 17) and 5-GTACTCAGCGCCAGCATCG-3′ (SEQ ID NO: 18) for GAPDH. qRT-PCR primers to detect the wild-type transcript levels in stable sgRNA lines were designed to have their 3′ ends around the Cas9 cut-site of the genes of interest and were as follows: 5′-GCGCTCTTCAGATCCACGAG-3′ (SEQ ID NO: 19) and 5-CCCGTGTGTCATGTAGTGAAC-3′ (SEQ ID NO: 20) for SALL4, 5′-GCAGAGTGTTCCTGAAGGAA-3′ (SEQ ID NO: 21) and 5-CTCAGGTAAATCATCTACTTTCAGC-3′ (SEQ ID NO: 22) for DSCC1, 5′-GAGGAACGAGGTGTCGTTTG-3′ (SEQ ID NO: 23) and 5-CACGTGGTAAACAGATCAGA-3′ (SEQ ID NO: 24) for SEPHS1, 5′-GCACTGGCGGTGTCAAGCCC-3′ (SEQ ID NO: 25) and 5-ATAAGTGGACCGTGAGATGC-3′ (SEQ ID NO: 26) for VRTN, 5′-ACAGCCACACACTACATCAG-3′ (SEQ ID NO: 27) and 5-TTGTGACGATCTCCAATTCC-3′ (SEQ ID NO: 28) for PIK3CA and 5′-GCAGTTTGCTCCGGAATATG-3′ (SEQ ID NO: 29) and 5-GCTGGCCAGCACAGACGCTG-3′ (SEQ ID NO: 30) for PDIA4.

### TRA-1-60 Immunocytochemistry

hESCs were trypsinized with TrypLE Select. Cells were collected in cold PBS containing 10% KSR, centrifuged at 300 g for 5 min and resuspended in 200 μl PBS containing 10% KSR. PE-conjugated mouse anti-human TRA-1-60 antibody (1:40, BD Biosciences) was then incubated with the cells for 30 min at 4° C. Cells were washed with PBS containing 10% KSR twice, centrifuged at 300 g at 4° C. and resuspended in PBS with 10% KSR. Immunolabelled cells were filtered through a 70 μm cell strainer and analysed in BD FACSAria III for the proportion of TRA-1-60-positive cells.

### Apoptosis Assay

hESCs were trypsinized gently with TrypLE Select. Apoptotic cells were labelled with Annexin V and propidium iodide (PI) using the MEBCYTO Apoptosis Kit according to the manufacturer's instructions (MBL). Labelled cells were filtered through a 70 μm cell strainer and analysed in BD FACSAria III for the proportion of annexin V-positive cells.

### PI Staining

PI staining was performed as described in Sagi, I., Egli, D. & Benvenisty, N. Identification and propagation of haploid human pluripotent stem cells. Nat. Protoc. 11, 2274-2286 (2016). Briefly, hESCs were trypsinized with TrypLE Select and fixed with cold methanol (4° C.). Fixed cells were treated with 200 μg ml⁻¹ RNase A (Sigma) for 30 min and stained with 50 μg ml⁻¹ PI for 5 min. Stained cells were filtered through a 70 μm cell strainer and analysed in BD FACSAria III for their cell-cycle profile.

### Western Blotting

hESCs were washed with PBS, lysed in sample buffer (100 nM Tris at pH 6.8, 200 mM dithiothreitol (DTT), 4% SDS, 0.2% bromophenol blue, 20% glycerol) and boiled for 5 min. Total protein originating from an equal number of cells was separated by 12% SDS-PAGE and transferred to a nitrocellulose membrane (Pall Corporation). Membranes were blocked with 8% BSA for 1.5 h at room temperature, sliced into strips for each primary antibody at the corresponding molecular weight ranges, and incubated with the primary antibodies (in TBS-T with 5% BSA) overnight at 4° C. Membranes were washed three times with TBS-T and incubated with the secondary antibody (in TBS-T with 5% BSA) for an hour at room temperature. Following the three washes with TBS-T, membranes were incubated with EZ-ECL (Biological Industries, cat. no. 20-500-120). Signals were detected using X-ray films (Fujifilm, cat. no. 47410). Working dilutions of the primary and secondary antibodies were as follows: anti-phospho-AKT antibody at 1:1,000 (Cell Signaling Technology, cat. no. 4060), anti-GAPDH antibody at 1:30,000 (Cell Signaling Technology, cat. no. 21185), anti-phospho-4E-BP1 antibody at 1:1,000 (Cell Signaling Technology, cat. no. 23684) and anti-rabbit-HRP at 1:5,000 (Santa Cruz Biotechnology, cat. no. SC-2004).

### Data Reporting

No statistical methods were used to predetermine the sample size. The investigators were not blinded to allocation during experiments and outcome assessment.

### Statistics and Reproducibility

Statistical analysis was performed using Python, R and Microsoft Office Excel.

Data are presented as mean-centred and with the standard error of the mean. An unpaired two-tailed t-test was performed for comparisons of two groups unless otherwise stated.

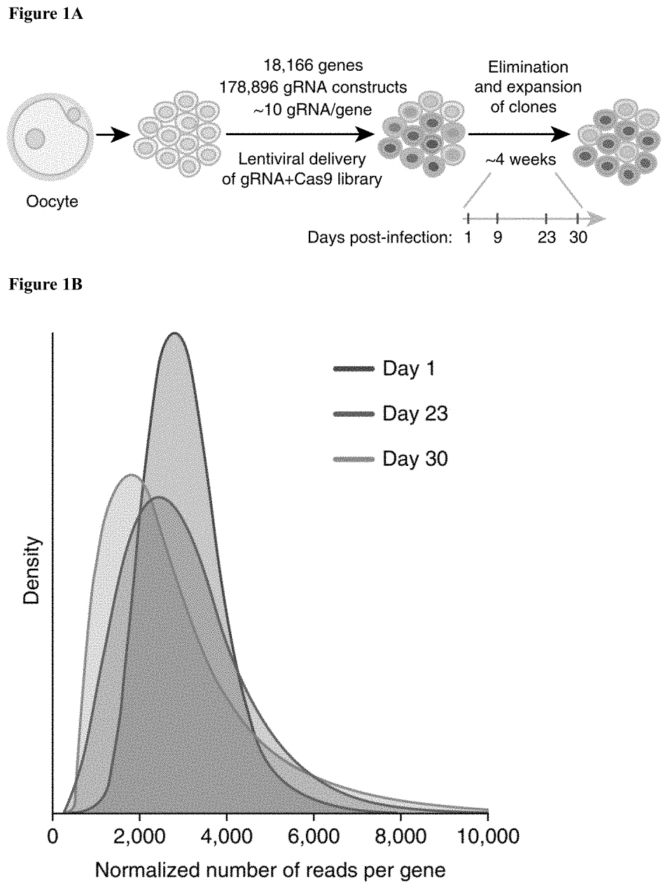

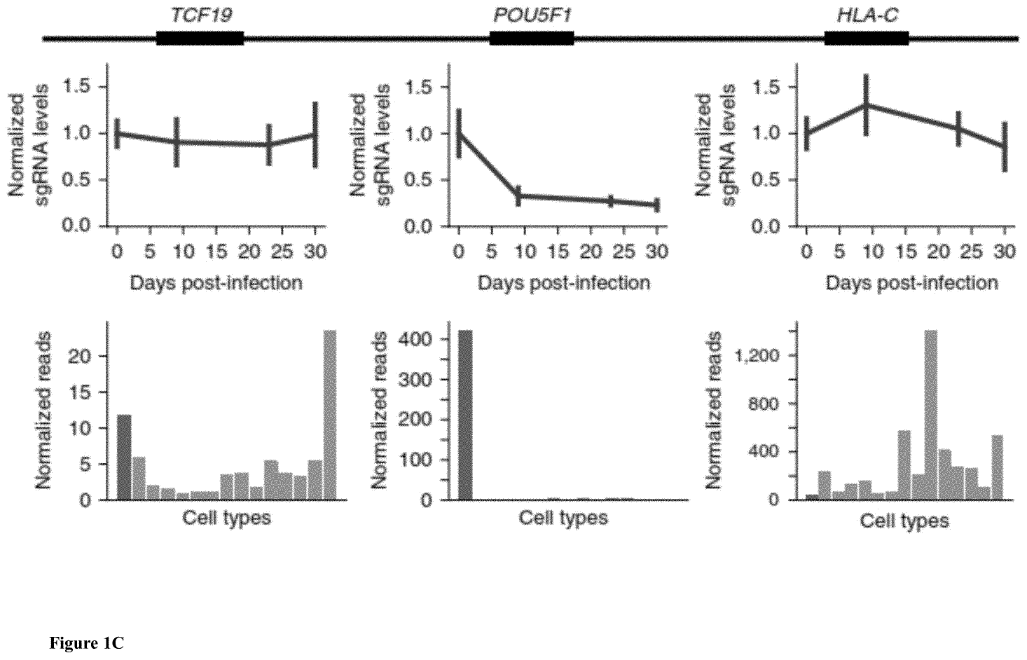

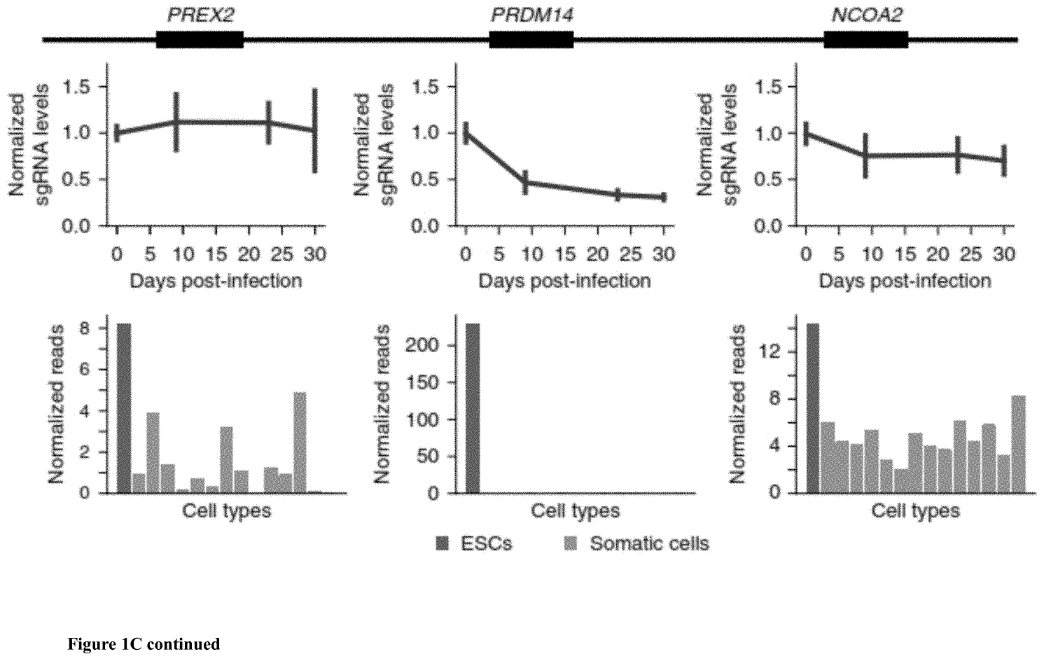

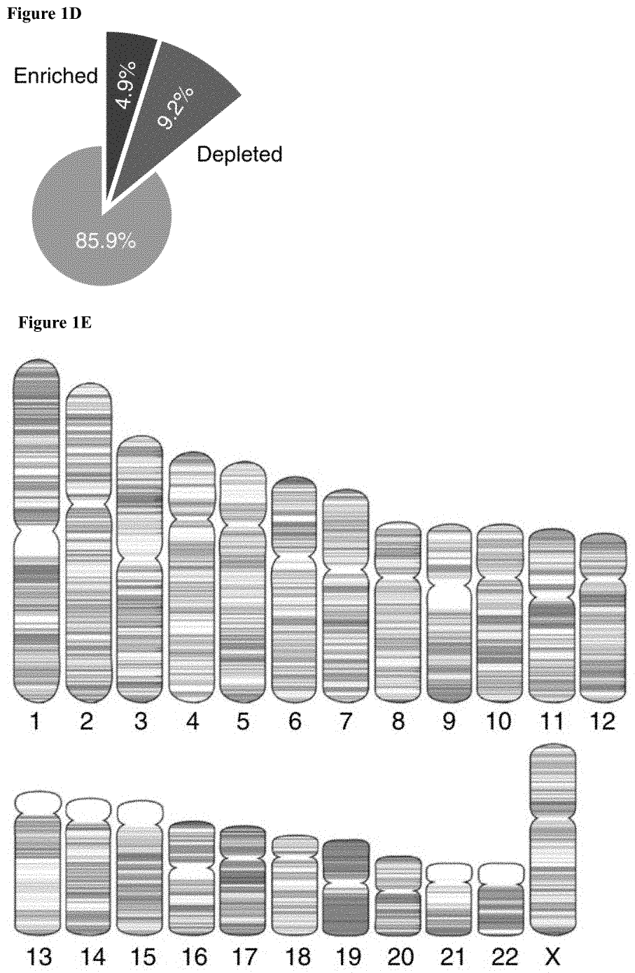

FDR was controlled using the Benjamini-Hochberg correction using P<0.05 as statistical significance. The exact P values and number of replicates per condition are stated in the figure legends with the statistical method used. Data presented in FIGS. 1B-1E, 2A-2F, 3A-3D, 5A-5B, 7A and D, 8A-8D, 9A-9B and 9D-9G, 10A-10B and 11 were derived by averaging two independent genome-wide screens with strongly correlated results (r=0.88, FIG. 7C). All other experiments were repeated with at least three independent biological repeats, unless otherwise stated.

### Data Availability

RNA-seq data that support the findings of this study have been deposited in the Gene Expression Omnibus (GEO) under accession codes GSE103846 and GSE107965. DNA-seq data that support the findings of this study have been deposited in the GEO database under accession code GSE111309. Previously published sequencing data that were re-analysed here are available under accession codes GSE62772, GSE73211, GSE80264 and GSE81791 and from the GTEx Portal version V6p at gtexportal.org/home/datasets. Registered users can access the files using the dbGaP accession no. phs000424.v6.p1.

### Example 1: Identification of Cell-Essential Genes in Human Pluripotent Stem Cells

To define the essentialome of hPSCs, we took advantage of our recent discovery of haploid hESCs to build a CRISPR-Cas9-based genome-wide loss-of-function mutant library (FIG. 1A). We utilized a human activity-optimized sgRNA library that targets more than 18,000 coding genes and contains 10 sgRNAs for more than 99% of the target genes. Using this library of about 180,000 gRNAs, we aimed to identify mutations in essential genes that affect the survival or normal growth of hESCs based on their depletion in the hESC population, as well as mutations in growth-restricting genes that provide growth advantage to hESCs based on their enrichment over time in culture. We analyzed the abundance of sgRNAs within the haploid hESC population at multiple time points after the co-delivery of sgRNAs and Cas9, and found gradual depletion and enrichment of numerous sgRNAs (FIGS. 1B and 7A). This observation allowed us to analyze two opposing subsets of genes, namely the essential and the growth-restricting genes. To assess the validity of our screen in the context of pluripotency, we followed the temporal changes in sgRNA representation for two well-characterized hESC-enriched and pluripotency-associated genes, POU5F1 (also known as OCT4) and PRDM14, as well as their neighboring hESC-expressed genes (FIG. 1C). sgRNAs targeting both POU5F1 and PRDM14 became significantly depleted within three weeks after the delivery of sgRNAs. In contrast, sgRNAs targeting the neighboring genes, which are not expressed exclusively in hESCs, were not depleted over time. To reveal significant changes in sgRNA representation between the initial and final hESC populations, we calculated a CRISPR score as the ratio of sgRNA abundance between final and initial populations for each gene (FIG. 7B). CRISPR scores demonstrated a high degree of reproducibility across replicate experiments (FIG. 7C). Based on this analysis we identified about 9% of the genes in the coding genome as essential for normal growth of hESCs, as well as about 5% of the genes as growth-restricting genes (FIG. 1D). Importantly, both essential and growth-restricting genes are distributed across all chromosomes without enrichment in specific chromosomal regions (FIGS. 1E and 7D).

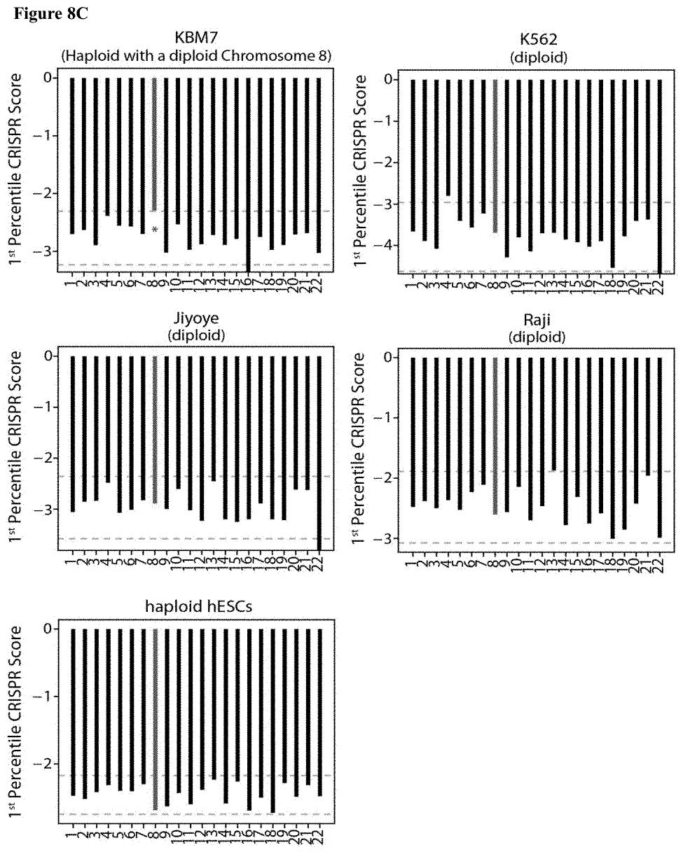

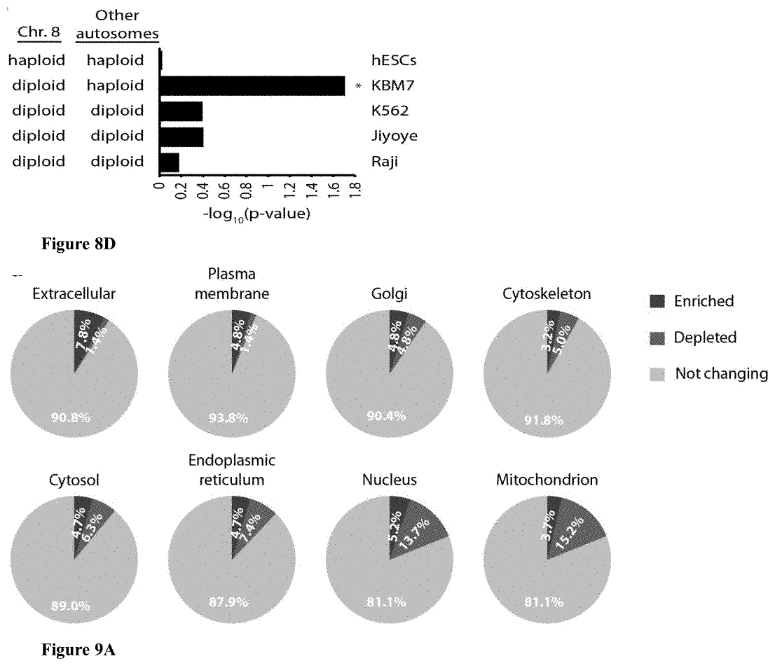

We then compared our list of essential genes to those identified in three previous screens performed in human cancer and immortalized lines using a variety of methodologies. We found a considerable overlap between the different screens, although each study also pointed to a unique set of essential genes (FIG. 8A). Clustering these datasets via a principal component analysis (PCA) revealed that they are separated mainly based on mutagenesis methodology, as recently suggested by others (FIG. 8B). Thus, in our comparisons, we focused on the cancer lines that were screened for essential genes using the same sgRNA library. Interestingly, even though the essentialome identified in hESCs clustered more closely to that of cancer lines defined using the same sgRNA library, a third of the essential genes identified in hESCs were unique to these cells, indicating that cell identity is also an important factor in shaping the gene essentiality landscape. Although genetic screens using the CRISPR-Cas9 technology have been efficiently performed in diploid cells, the use of haploid cells further increases the efficiency of generating complete loss-of-function frameshift mutations (see “Data Analysis” section in Materials and Methods, and FIGS. 8C-8D).

### Example 2: Cellular Localization and Disease-Association of Cell-Essential Genes

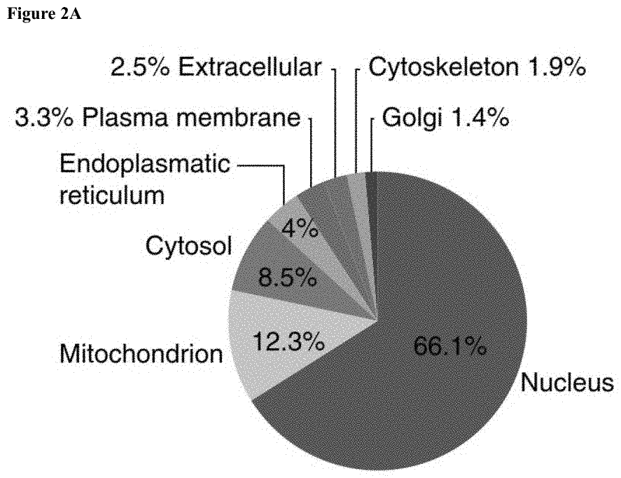

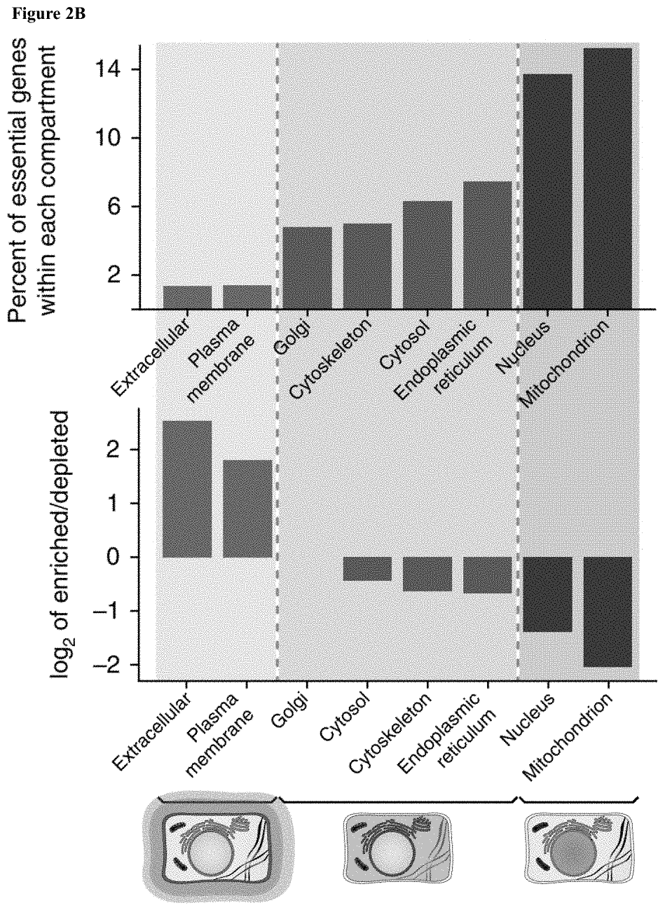

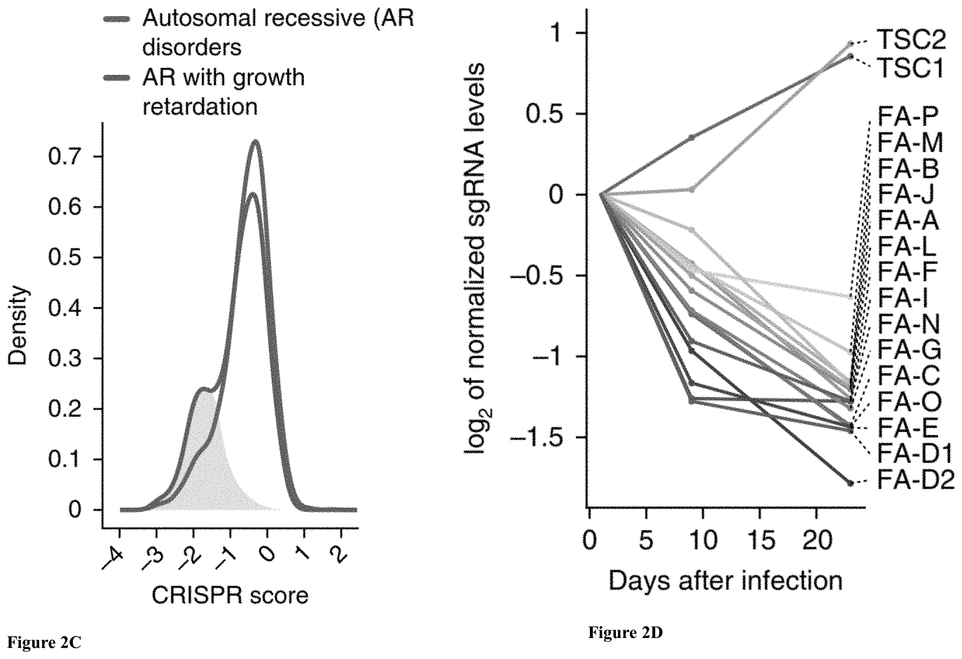

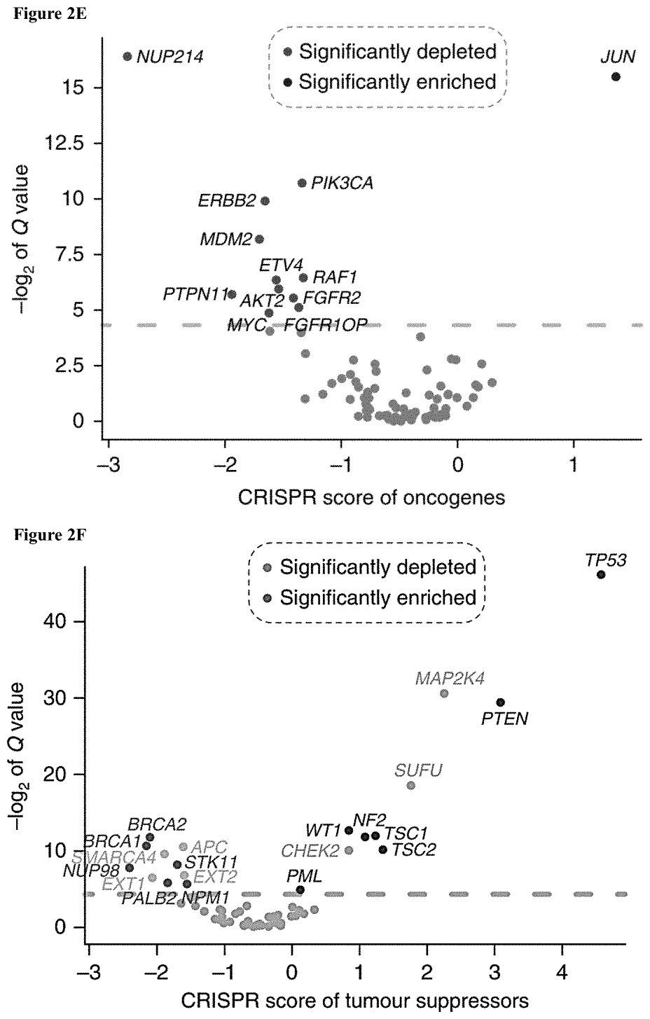

We next investigated different aspects relating to cell-essential genes in the context of hESCs, including their cellular localization and their association with autosomal recessive (AR) human genetic disorders and tumor-causing mutations. We found that 66% of the cell-essential genes encode proteins that localize to the nucleus, 12% encode mitochondrial proteins and 8.5% encode cytosolic proteins, while the rest encode proteins that are distributed between the endoplasmic reticulum, plasma membrane, extracellular space, cytoskeleton and the Golgi (FIG. 2A). Analyzing the proportion of essential genes among all genes associated with each of these eight cellular compartments revealed three categories: (a) compartments related to the extracellular space showed low proportions of essential genes (about 1%); (b) compartments related to the cytoplasm showed medium proportions of essential genes (5-7%); and (c) the nuclear and mitochondrial compartments showed high proportions of essential genes (14-15%) (FIG. 2B, upper panel). This bias in the cellular localization landscape of the essentialome may suggest different roles for essential genes in the regulation of cell-growth and/or different levels of functional redundancy in the various compartments. Interestingly, when we examined the ratio between the number of growth-restricting genes and essential genes within each compartment, we observed that the extracellular space and the plasma membrane had higher representation of growth-restricting genes compared with essential genes, as opposed to the nucleus and mitochondrion (FIG. 2B, lower panel, and FIG. 9A), which may hint at the important involvement of the cellular environment in inhibiting cell-growth. The differences of proportion of essential genes among compartments was equally apparent when only expressed genes (FPKM>1) were analyzed (FIG. 9B). An analysis of the fraction of essential genes in different cellular compartments in leukemic KBM7 cells demonstrated a very similar pattern to that observed in hESCs, suggesting that this distribution pattern is shared across different cell types (FIG. 2B, upper panel and FIG. 9C).

Many of the genes analyzed in our screen also underlie human genetic disorders and are mutated in patients. We speculated that some of the genes carrying mutations associated with AR human disorders could be important for the normal growth of hESCs, and hence potentially affect growth in the early human embryo. Of 2,099 human AR-related genes reported in the Online Mendelian Inheritance in Man (OMIM) database that were also represented in our library, 226 (10.8%) were found to be essential for hESC-growth. Interestingly, genes responsible for AR disorders that exhibit a growth-retardation phenotype were significantly enriched in essential genes (154 out of 766, 20.1%, P<0.001) (FIG. 2C). A similar analysis in the near-haploid leukemic KBM7 cell line failed to demonstrate a significant enrichment of essential genes among the genes causing autosomal recessive disorders with growth-retardation phenotypes in these cells (FIG. 9D), suggesting that hESCs provide a more suitable model to study the phenotypes of developmental human disorders. Among AR disorders with a growth-retardation phenotype, we focused on Fanconi anemia (FA), which was reported difficult to model in hPSCs as the growth of the mutant cells was compromised. 14 of 15 genes associated with mutations causing FA were identified as essential in hESCs (FIG. 2D). In contrast, TSC1 and TSC2, two genes with autosomal dominant mutations associated with tuberous sclerosis and overgrowth in multiple tissues, were identified as growth-restricting genes in these cells (FIG. 2D). Our analysis suggests that the phenotype of growth-retardation associated with AR disorders may initiate, in one fifth of the disorders, at very early stages of embryogenesis. These findings open up an exciting future direction towards modeling growth-retardation phenotypes already in hPSCs for a wide group of AR disorders.

Next, we analyzed canonical oncogenes and tumor suppressor genes in terms of their essentiality and growth-restriction in the context of hESCs. Nearly all oncogenes whose mutations significantly affected the growth of hESCs were classified as essential for normal growth, with the exception of JUN which was found to be growth-restricting (FIGS. 2E and 9E). Indeed, c-Jun was shown to interfere with the induction of pluripotency in mouse cells. In contrast, tumor suppressors were divided into essential and growth-restricting gene classes (FIGS. 2F and 9F). Gene Ontology analysis revealed that growth-restricting tumor suppressors were enriched in apoptosis-related genes (FIG. 2F, dark blue points), whereas essential tumor suppressor genes were enriched in processes such as genomic instability and DNA-repair (FIG. 2F, dark red points). This analysis thus points to distinct roles for tumor suppressor genes in hPSCs.

A comparison of growth-restriction by tumor suppressors and essentiality of oncogenes between hESCs and four cancer cell lines demonstrated that the genetically aberrant lines show marked variation in these genes (FIG. 3G). This comparison yielded three groups of genes: 1) Genes that were growth-restricting in hESCs but lost this feature in aberrant cells (FIG. 9G, left heatmap); 2) Genes that are essential in hESCs but lost their essentiality in aberrant lines (FIG. 9G, middle heatmap), and 3) Genes that were not essential in hESCs but became essential for growth in cancer cells (FIG. 3G, right heatmap).

### Example 3: Identification and Characterization of hESC-Essentialome

The pluripotent state is governed by a set of genes whose expression is enriched in hESCs. Therefore, we hypothesized that hESC-essential genes would be more prevalent within hESC-enriched genes. To test this hypothesis, we performed a gene expression-based analysis in which we divided the genes represented in the library into subcategories related to their expression and enrichment in hESCs. The CRISPR score distribution of hESC-expressed genes shifted towards more depleted values as compared with the distribution of genes that are not expressed (or expressed at low levels) in hESCs (FIG. 3A). The percentage of essential genes increased from 9.2% among all genes to 13.5%0 among hESC-expressed genes, to 19.8% among genes enriched in hESCs and up to 22.7% among nuclear hESC-enriched genes (FIG. 3A). This stepwise analysis led to the identification of a subset of hESC-enriched genes, constituting the hESC-essentialome. Of these genes, 67% are nuclear, 18% localize to cytosol and the rest is distributed across the cytoskeleton, plasma membrane, extracellular space and Golgi (FIG. 3B). Importantly, the hESC-specific essentialome is significantly depleted of genes localized to mitochondria (HG-test, n=50 genes, P=12e⁻⁴), even though this compartment was significantly enriched among the cell-essential genes (FIG. 2A). Functional categorization of the hESC-specific essentialome revealed that the majority of genes are related to two main functional groups: cell-cycle and DNA-repair (˜53%) and transcription (21%) (FIG. 3C).

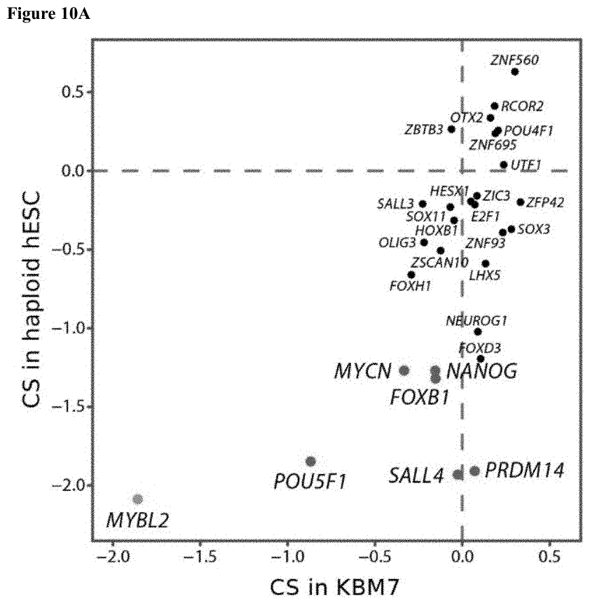

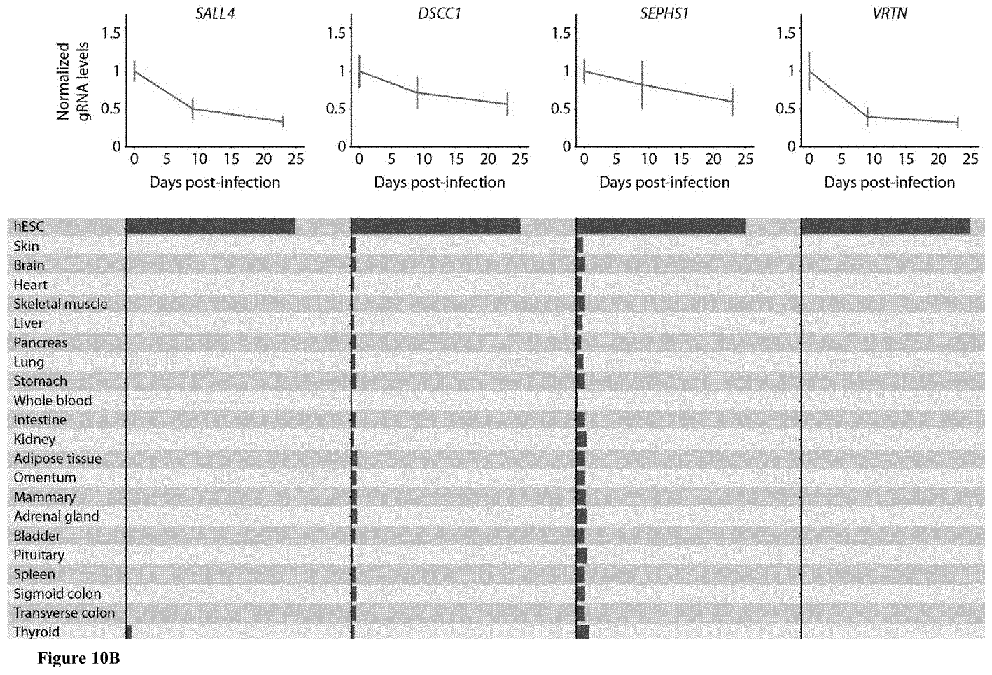

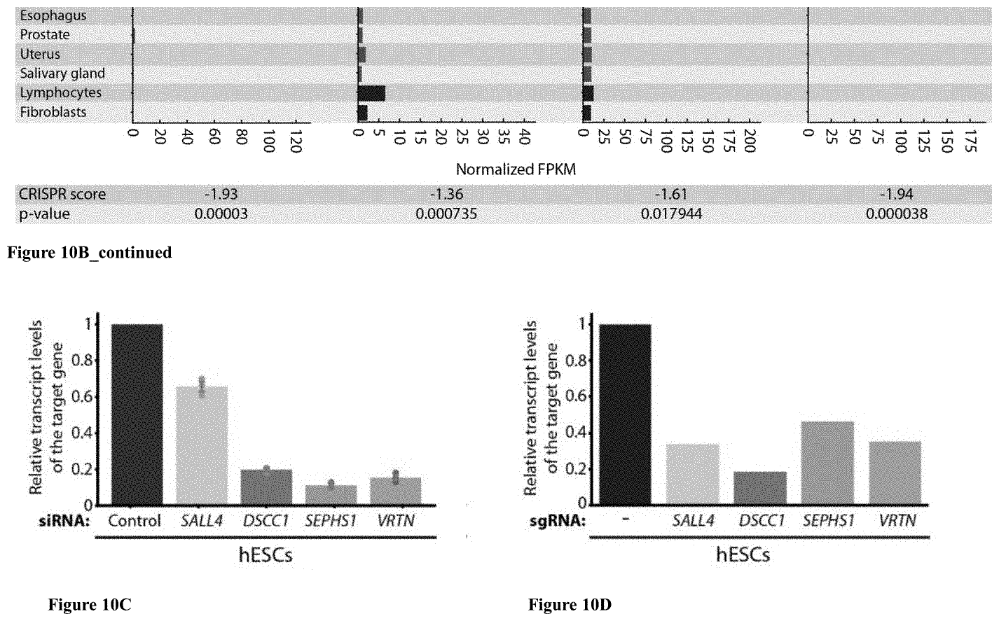

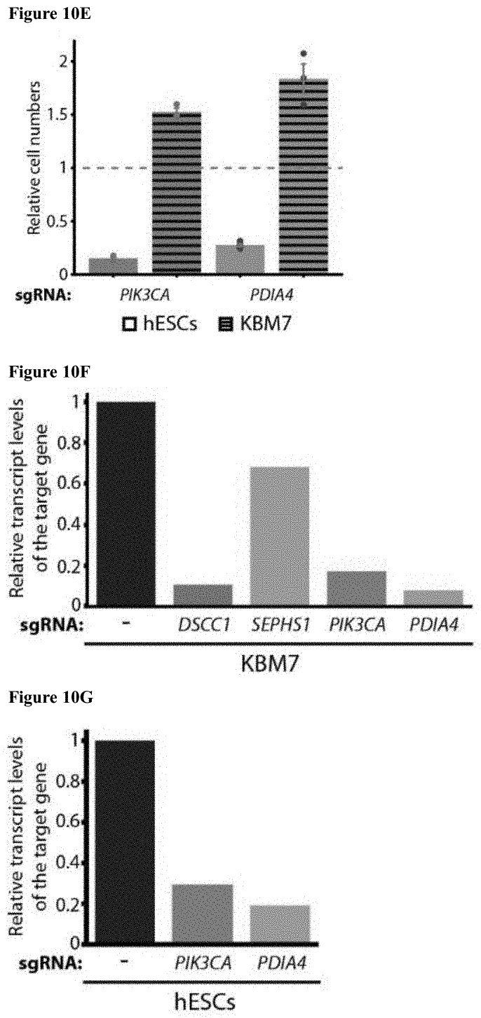

Transcription factor (TF) networks have been classically studied within the context of pluripotency. Therefore, of ˜2,000 annotated human TFs, we focused on those that showed enriched expression in hESCs and analyzed their CRISPR scores. We found that the hESC-essential TFs include well-characterized pluripotency factors such as SALL4, POU5F1, PRDM14 and NANOG, as well as MYBL2, FOXB1 and MYCN, but not pluripotency-associated factors such as UTF1 and ZFP42 (FIG. 3D). Interestingly, we identified a subset of growth-restricting TFs such as ZNF560, RCOR2, OTX2 and ZNF695. A comparison between hESCs and the leukemic KBM7 cells for these hESC-essential TFs revealed that these TFs were indeed essential exclusively in hESCs, with the exception of MYBL2 (FIG. 10A). We reasoned that the essentiality difference between the essential and dispensable hESC-enriched TFs might be due to their expression levels in hESCs as compared with immediate progenitor cells. To test this, we compared the expression of TFs in hESCs with that in embryoid bodies (EBs) that had undergone differentiation for 20 days (FIG. 3E). Indeed, the EB/hESC expression ratio was found to be smaller for the essential hESC-enriched TFs, suggesting that cell-type-specific gene expression is an important factor in determining essentiality.

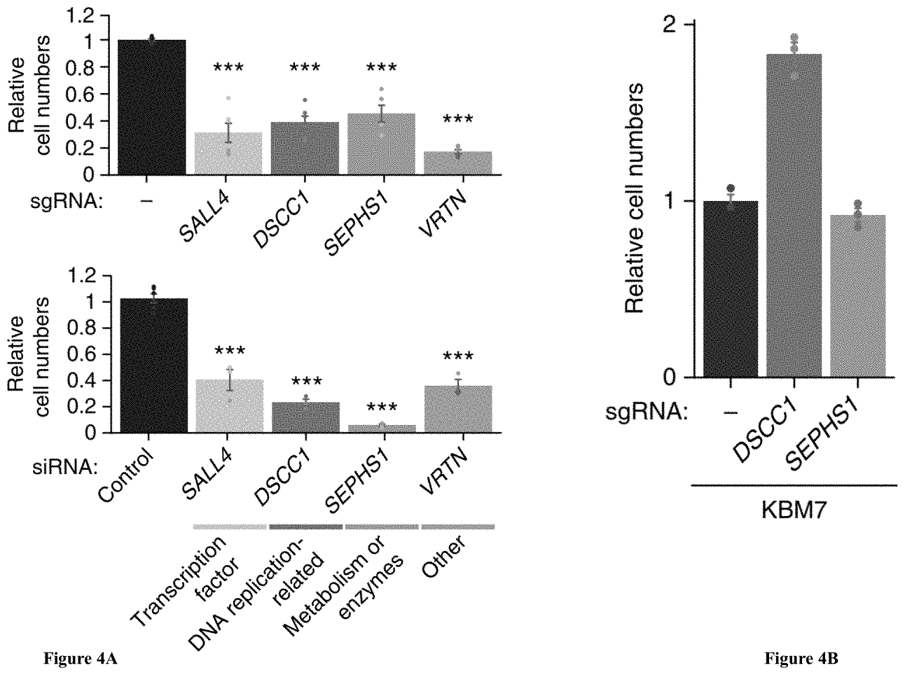

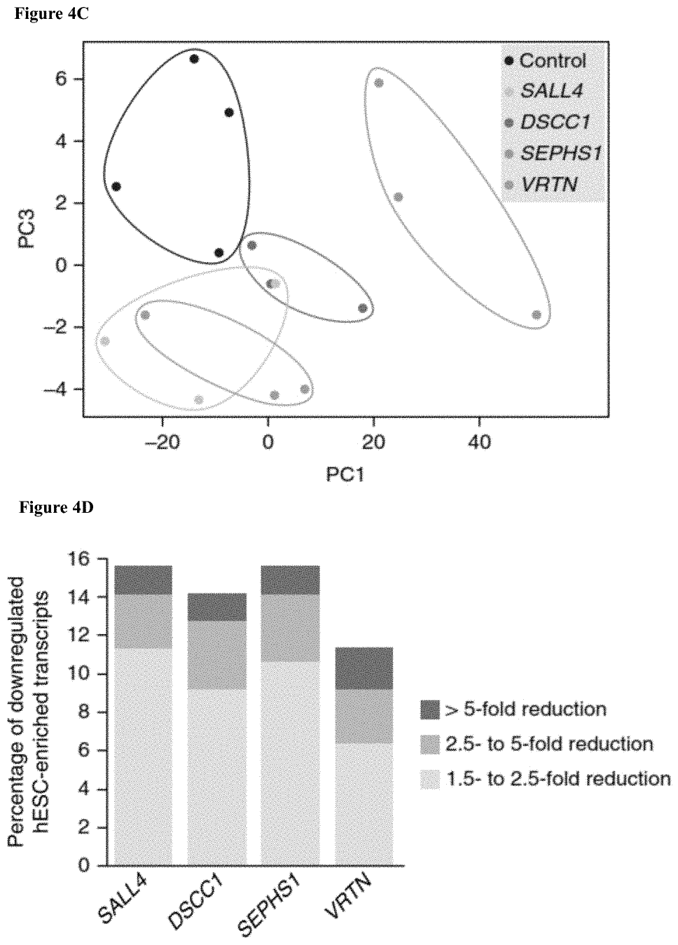

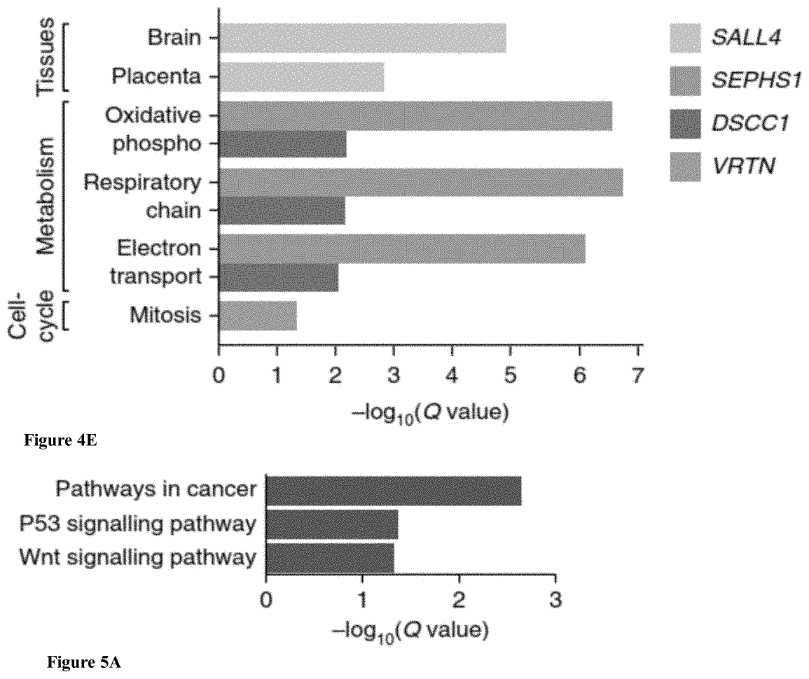

Next, we aimed to validate the hESC-essentialome we defined in haploid hESCs. To this end, we used both RNA interference (RNAi) and CRISPR-Cas9 mutagenesis in diploid hESCs, focusing on genes that represent different functional categories. siRNA-mediated knockdown, as well as sgRNA-mediated knockout, of the pluripotency-associated TF SALL4, the DNA replication factor DSCC1, the selenium metabolism enzyme SEPHS1 and the putative DNA-binding, nuclear protein VRTN inhibited the growth of normal diploid hESCs (FIGS. 4A and 10B). Reduction in transcript levels in the siRNA and sgRNA experiments was confirmed for these four genes (FIGS. 10C-10D).

To demonstrate the concept of hESC-specific essential genes, we compared the growth rates of hESCs and the near-haploid leukemic KBM7 cells mutated in genes that were found as essential in hESCs. We thus chose to analyze four genes that are expressed in both cell types: DSCC1 and SEPHS1 (FPKM values were 9.1 and 5.6 for KBM7 and 40.7 and 203.1 for hESCs, respectively), discussed above, alongside two additional genes, the oncogene PIK3CA and the endoplasmic reticulum gene PDIA4 (FPKM values were 5 and 59.7 for KBM7 and 4.4 and 214.8 for hESCs, respectively). Mutations in either of these genes did not perturb the growth of KBM7 cells, while significantly inhibiting the growth of hESCs (FIGS. 4B and 10E-10G).

We then aimed to unravel some of the pathways affected by hESC-essential genes. We thus analyzed the transcriptome of cells with knockdown in SALL4, DSCC1, SEPHS1 or VRTN. Principal component analysis (PCA) based on RNA sequencing demonstrated that inhibition of expression of these genes caused separation from the control siRNA-treated conditions at different degrees, suggesting distinct functions for these genes (FIG. 4C). Importantly, 12-16% of the hESC-enriched genes were downregulated upon knockdown of each of these four genes (FIG. 4D). Gene ontology (GO) analysis revealed that hESC-essential genes affect different aspects of hESC biology: SALL4 knockdown upregulated genes related to differentiation to ectodermal brain and trophectodermal placenta cells, suggesting that its inhibition induced differentiation of the pluripotent cells. Interestingly, knockdown of either SEPHS1 or DSCC1 affected energy metabolism through oxidative phosphorylation, whereas VRTN knockdown showed a modest effect on mitosis (FIG. 4E).

### Example 4: Analysis of Growth-Restricting Genes Highlights the Role of the P53-mTOR Pathway in hESC-Growth

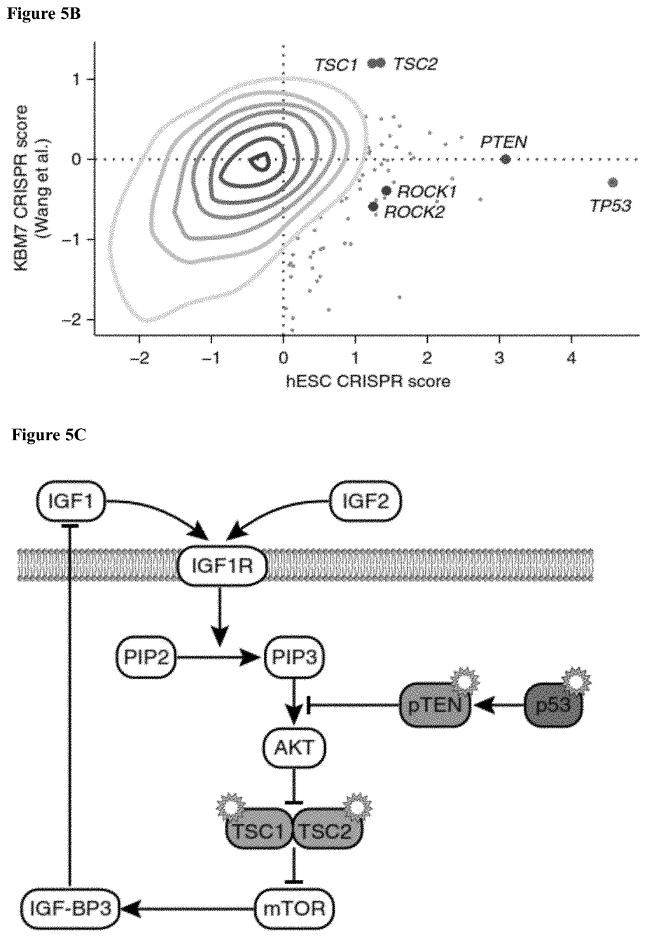

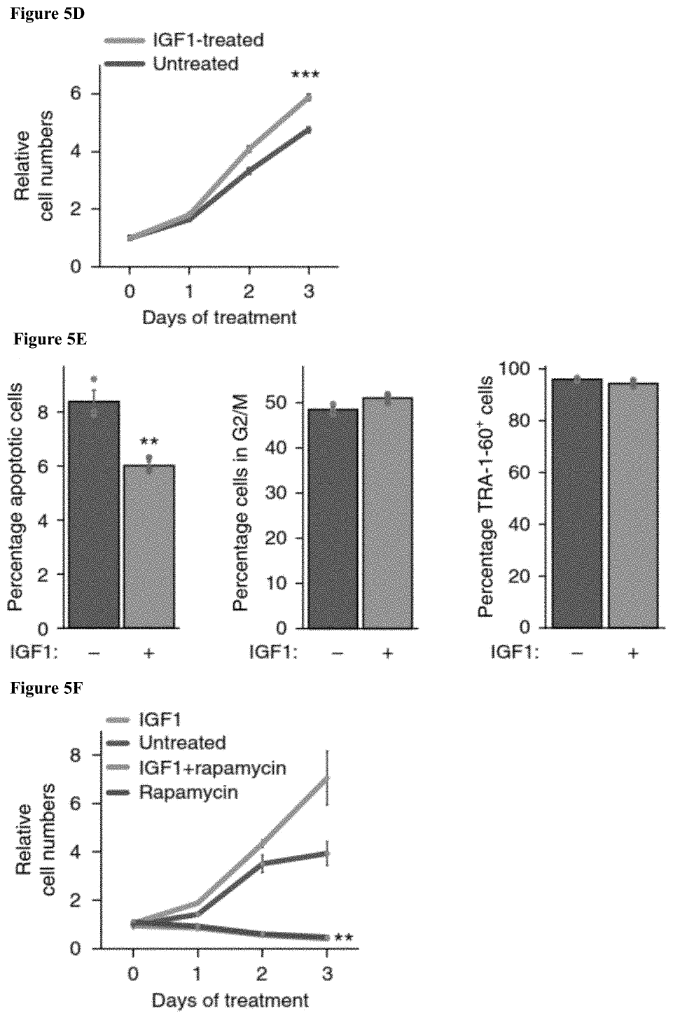

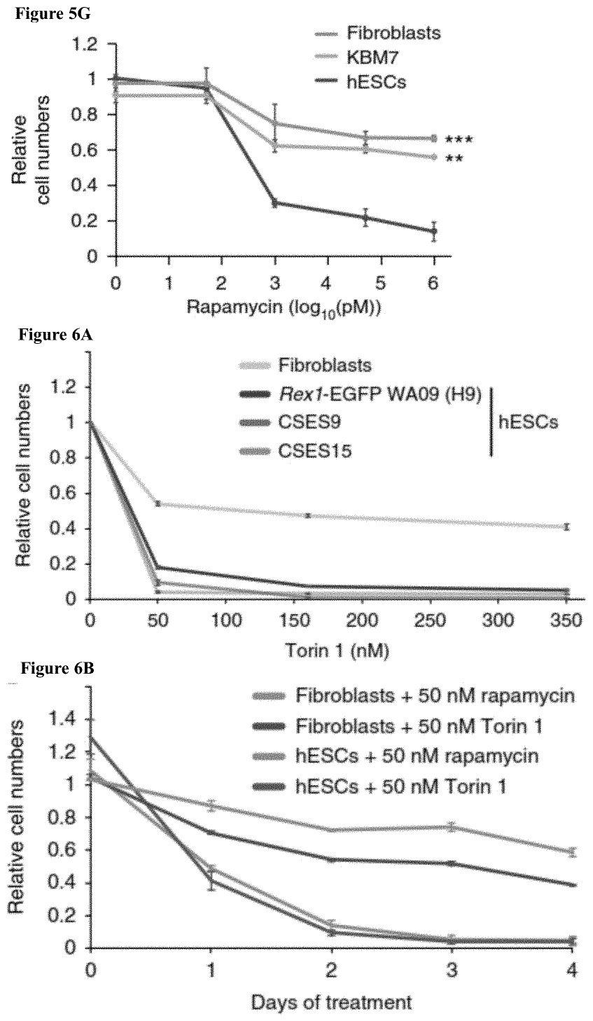

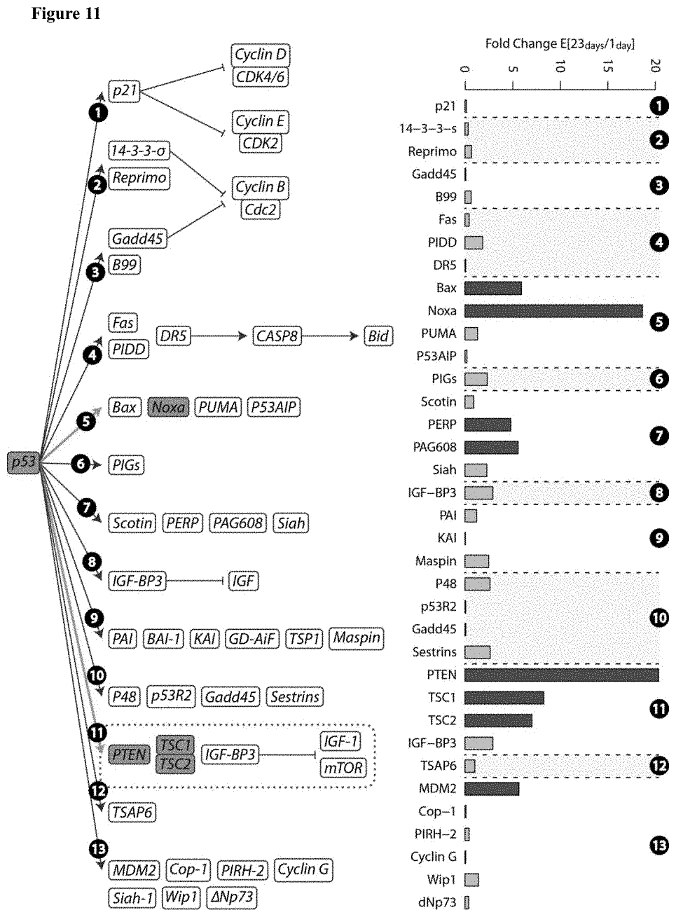

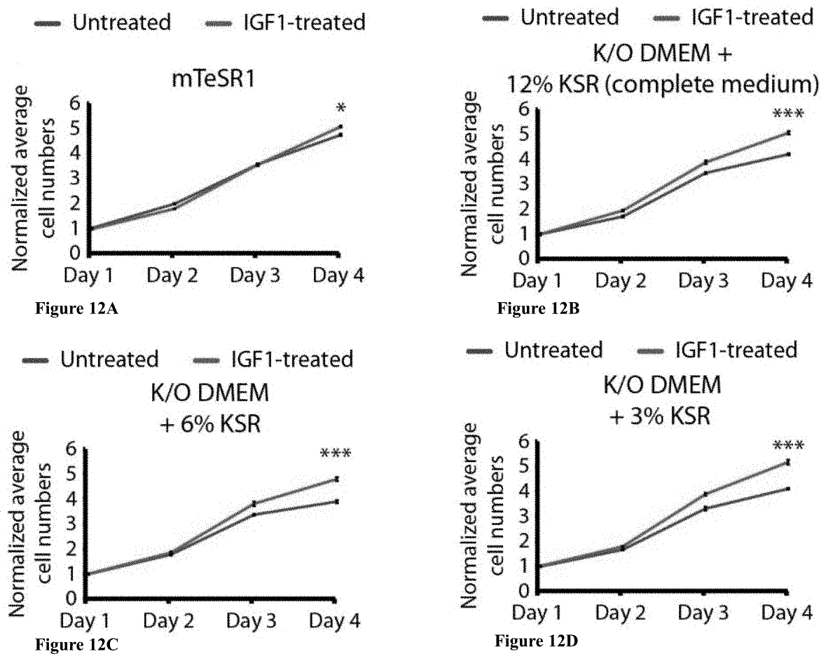

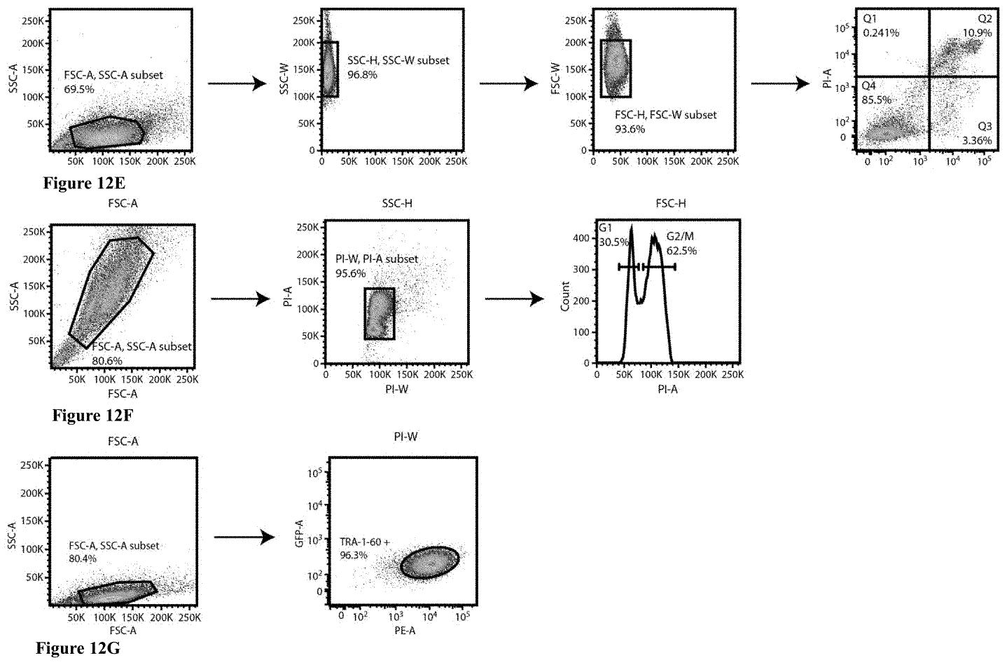

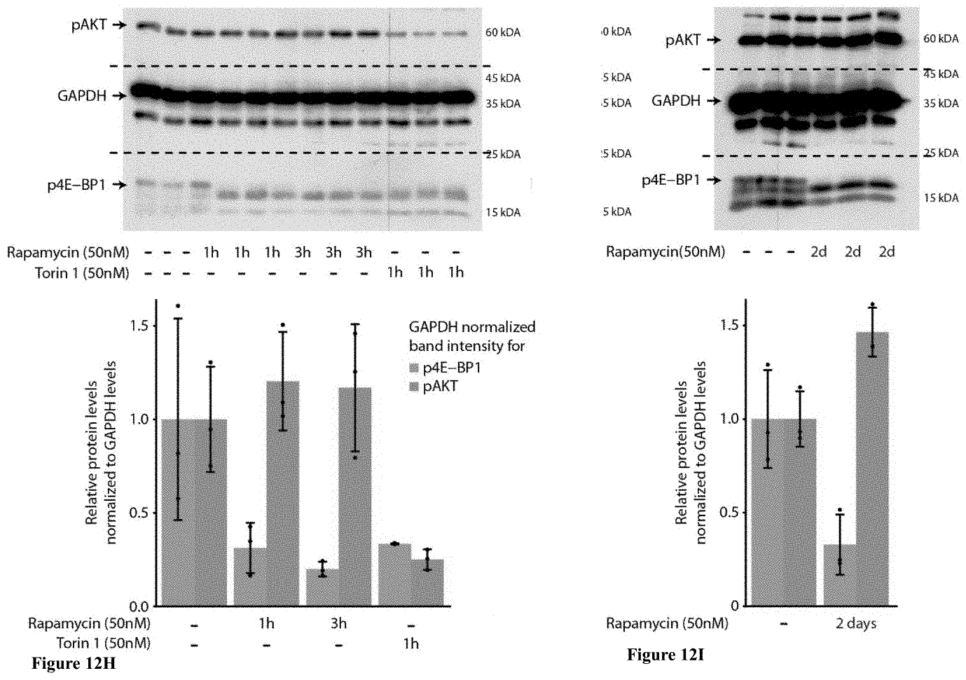

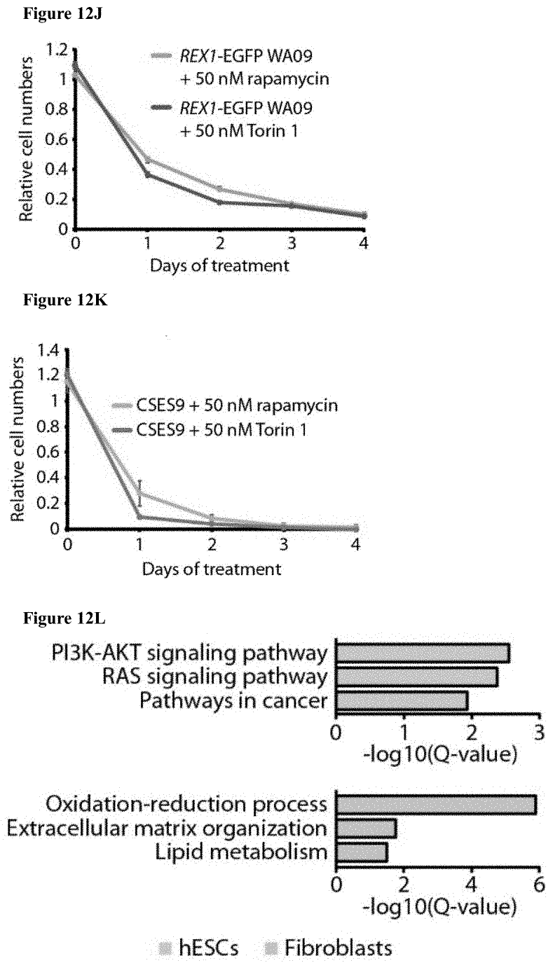

We next analyzed the group of growth-restricting genes in hESCs. GO analysis of the highest scoring 50 growth-restricting genes showed enrichment in pathways related to cancer, the P53 signaling pathway and WNT signaling pathway (FIG. 5A). Importantly, the tumor suppressor genes TP53 and PTEN were identified as the highest scoring growth-restricting genes. We then compared the CRISPR scores of all genes in our hESC screen to those in a previous screen performed in the near-haploid leukemic cell line KBM7 (FIG. 5B). This analysis demonstrated that the members of P53 and ROCK pathways, which were identified among the highest scoring growth-restricting genes in hESCs, were absent among the growth-restricting genes in KBM7 cells, suggesting that the P53 pathway may already be mutated in this cancer cell line. Interestingly, among 13 distinct P53 target pathways, we found an enrichment of highest scoring growth-restricting genes of hESCs in the branch inhibiting the IGF1/mTOR pathway (FIGS. 5C and 11). Therefore, we aimed to validate the role of this pathway in the regulation of hESC growth. We found that especially under conditions with low levels of KSR, addition of IGF1 significantly increased the growth rate of diploid hESCs (FIGS. 5D and 12A-12D). We reasoned that the IGF1-mediated increase in growth rate could be attributed to the regulation of cell death through apoptosis, to regulation of proliferation rate through changes in cell-cycle or to differences in spontaneous differentiation. Therefore, we measured the percentage of apoptotic cells, cells in G2/M phase and TRA-1-60⁺-pluripotent cells after IGF1-treatment, and found that IGF1 significantly decreases the percentage of apoptotic hESCs while not affecting their cell-cycle nor differentiation dynamics (FIGS. 5E, 12E-12G). To demonstrate the direct involvement of mTOR in the growth-regulation of hESCs, we treated hESCs with rapamycin, a selective inhibitor of mTOR, in the presence and absence of IGF1. Inhibition of mTOR caused drastic growth-inhibition of hESCs, and IGF1 failed to rescue this inhibition, suggesting that IGF1 acts upstream of mTOR (FIG. 5F).

### Example 5: hESCs are More Sensitive to Growth-Regulation by the mTOR Pathway Compared with Somatic Cells

Interestingly, hESCs were more sensitive to growth-inhibition under various doses of rapamycin as compared to human foreskin fibroblasts, as well as KBM7 cells, suggesting that the IGF1/mTOR pathway regulates hESC-growth in a cell type-selective manner (FIG. 5g). mTOR kinase is associated with two major complexes, namely mTOR complexes 1 and 2 (mTORC1 and mTORC2). Rapamycin was shown to inhibit mTORC1 but not mTORC2 (FIG. 12H-12I). To assess the contribution of these complexes to the selective sensitivity of hESCs for mTOR pathway, we utilized a catalytic inhibitor of mTOR, Torin 1, which can inhibit both mTORC1 and mTORC2 (FIG. 12H). We found that Torin 1 treatment completely abolished the growth and survival of hESCs after two days of treatment at low nanomolar concentrations, whereas the growth of fibroblasts was inhibited up to 60% under the same conditions (FIG. 6A). Interestingly, inhibition of mTORC1 alone induced dramatic growth-inhibition in hESCs, whereas inhibition of both mTORC1 and 2 had only a partial effect on cell-growth in actively proliferating fibroblasts (FIGS. 6B and 12J-12K).

To unravel the molecular mechanism of the sensitivity of hESCs to mTORC1-inhibition, we analyzed the transcriptomes of rapamycin-treated hESCs and fibroblasts. We found that in hESCs, two days of rapamycin treatment downregulated genes associated with several growth- or apoptosis-related pathways such as RAS/PI3K-AKT/MAPK signaling pathways, P53 signaling pathway and amino acid biosynthesis pathways (FIG. 6C). In contrast, in fibroblasts rapamycin treatment did not significantly downregulate genes enriched in these pathways, but downregulated genes related to lipid metabolism (FIG. 6C). A similar difference in the downregulated pathways between hESCs and fibroblasts was also observed after four days of rapamycin treatment (FIG. 6L).

We next examined the effect of mTORC1 inhibition in hESCs at the cellular level, namely in the context of apoptosis, cell-cycle and differentiation. Rapamycin treatment increased the percentage of apoptotic hESCs (FIGS. 6D, and 12E), accompanied by an increase in the levels of TP53 (FIG. 6E). Interestingly, TP53 did not increase in rapamycin-treated fibroblasts. Rapamycin treatment also had a significant effect on the cell-cycle dynamics of hESCs by increasing the percentage of hESCs in G1 phase and decreasing the percentage of hESCs in G2/M phases (FIGS. 6F-6G, and 12F). This observation was supported by the decrease in expression of the G1-S transition factors CCND1 and CCND2 (FIG. 611). In contrast to hESCs, fibroblasts did not downregulate CCND1 and CCND2 upon rapamycin treatment. Although the major effects of mTORC1 inhibition were an increase in apoptosis and a decrease in proliferation, likely mediated through a G1-arrest, we also found that rapamycin treatment caused a modest decrease in the fraction of hESCs that were positive for the expression of the pluripotent cell marker TRA-1-60 (FIGS. 6I-6J, and 12G).

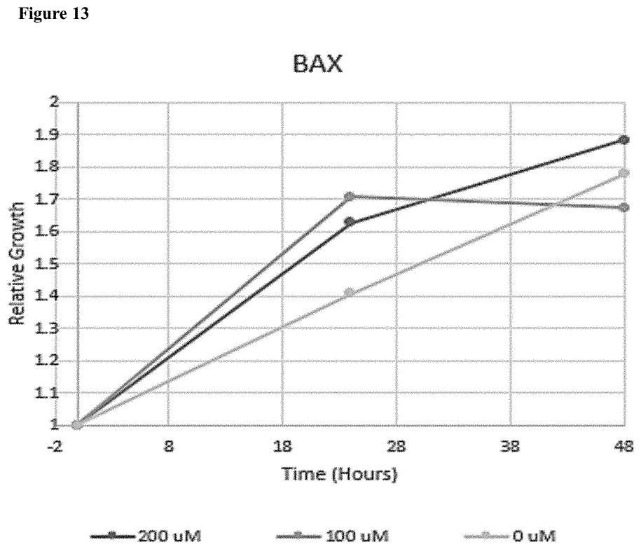

Next, we examined the effect of Bax inhibition on hESC growth. hESCs were treated with either 0, 100, or 200 uM of BAX-inhibiting peptide V5 (BIP-V5, Sigma). At time point zero when the peptide was added CellTiter-Glo reagent was also added to the cells and luminescence was measured with a plate reader. Bax inhibition increased the relative growth of the hESCs by 15-25% after 24 hours (FIG. 13).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method of maintaining or reducing the frequency of p53 mutation in a population of pluripotent stem cells (PSCs), the method comprising at least one of:
a. activating mTOR signaling in said population;
b. inhibiting Bax expression, function or both in said population;
c. inhibiting phorbol-12-myristate-13-acetate-induced protein 1 (Noxa), expression, function or both in said population;
d. inhibiting p53 apoptosis effector related to PMP22 (PERP) expression, function or both in said population; and
e. inhibiting zinc finger matrin-type 3 (PAG608) expression, function or both in said population;
thereby maintaining or reducing the frequency of p53 mutation in a population of PSC.

2. The method of claim 1, wherein said PSCs are selected from embryonic stem cells (ESCs) and induced pluripotent stem cells (iPSCs), optionally wherein said PSCs are human PSCs.

3. The method of claim 1, wherein said inhibiting comprises supplementing media comprising said hESCs with an inhibitor and said activating comprises supplementing media comprising said hESCs with an mTOR activator.

4. The method of claim 3, wherein said activator is a direct activator, said inhibitor is a direct inhibitor, or both, optionally wherein said mTOR activator is IGF1.

5. The method of claim 1, wherein said activating comprises inhibiting expression, function or both of an inhibitory protein of the mTOR pathway selected from PTEN, TSC1 and TSC2.

6. The method of claim 1, wherein said method does not comprises differentiation of said PSCs.

7. The method of claim 1, wherein said method does not increase differentiation of said PSCs as compared to control PSCs.

8. The method of claim 1, wherein said method does not decrease apoptosis in said population.

9. The method of claim 1, wherein said method increases the growth rate of said population.

10. The method of claim 1, further comprising using said population of PSCs in an assay requiring wild type p53 expressing cells.

11. A method of culturing a population of PSCs, the method comprising performing at least two of the following to said population in culture:
a. activating mTOR signaling in said population;
b. inhibiting Bax expression, function or both in said population;
c. inhibiting phorbol-12-myristate-13-acetate-induced protein 1 expression (Noxa), function or both in said population;
d. inhibiting p53 apoptosis effector related to PMP22 (PERP) expression, function or both in said population; and
e. inhibiting zinc finger matrin-type 3 (PAG608) expression, function or both in said population;
thereby culturing a population of PSC.

12. The method of claim 11, wherein said PSCs are selected from embryonic stem cells (ESCs) and induced pluripotent stem cells (iPSCs), optionally wherein said PSCs are human PSCs.

13. The method of claim 11, wherein said inhibiting comprises supplementing media comprising said hESCs with an inhibitor and said activating comprises supplementing media comprising said hESCs with an mTOR activator.

14. The method of claim 13, wherein said activator is a direct activator, said inhibitor is a direct inhibitor or both, optionally wherein said mTOR activator is IGF1.

15. The method of claim 11, wherein said activating comprises inhibiting expression, function or both of a protein of the mTOR pathway selected from PTEN, TSC1 and TSC2.

16. The method of claim 11, wherein said culturing
a. does not comprises differentiation of said hESCs;
b. does not decrease apoptosis in said population;
c. increases the growth rate of said hESCs; or
d. a combination thereof.

17. PSC media comprising at least one of:
a. a Bax inhibitor;
b. a Noxa inhibitor;
c. a PERP inhibitor; and
d. a PAG608 inhibitor.

18. The media of claim 17, further comprising an mTOR activator; optionally wherein said mTOR activator is insulin-like growth factor 1 (IGF1).

19. The media of claim 17, wherein said inhibitor
a. inhibits mRNA stability, mRNA translation or protein function; or
b. is a direct inhibitor of at least one of Bax, Noxa, PERP and PAG608.

20. The method of claim 17, wherein said PSC is selected from an ESC and an iPSC, optionally wherein said PSC is a human PSC.


SEQUENCE LISTING (30 sequences)

SEQ ID NO: 1 - DNA, 60 bp, Artificial
  Synthetic
tcgtcggcagcgtcagatgtgtataagagacagggctttatatatcttgtggaaaggacg

SEQ ID NO: 2 - DNA, 59 bp, Artificial
  Synthetic
gtctcgtgggctcggagatgtgtataagagacagacggactagccttattttaacttgc

SEQ ID NO: 3 - DNA, 20 bp, Artificial
  Synthetic
gcgctcttcagatccacgag

SEQ ID NO: 4 - DNA, 20 bp, Artificial
  Synthetic
gcagagtgttcctgaaggaa

SEQ ID NO: 5 - DNA, 20 bp, Artificial
  Synthetic
cacgtggtaaacagatcaga

SEQ ID NO: 6 - DNA, 20 bp, Artificial
  Synthetic
gcactggcggtgtcaagccc

SEQ ID NO: 7 - DNA, 20 bp, Artificial
  Synthetic
acagccacacactacatcag

SEQ ID NO: 8 - DNA, 20 bp, Artificial
  Synthetic
gctggccagcacagacgctg

SEQ ID NO: 9 - DNA, 20 bp, Artificial
  Synthetic
ttgaggggagatgggtactg

SEQ ID NO: 10 - DNA, 21 bp, Artificial
  Synthetic
aataagatggggacagggttg

SEQ ID NO: 11 - DNA, 20 bp, Artificial
  Synthetic
ttagccttccacccaaactg

SEQ ID NO: 12 - DNA, 19 bp, Artificial
  Synthetic
tcccaaagcgcatgtctac

SEQ ID NO: 13 - DNA, 20 bp, Artificial
  Synthetic
aggcattacccgtagtcgtg

SEQ ID NO: 14 - DNA, 21 bp, Artificial
  Synthetic
tccagaaaaccattcaagacg

SEQ ID NO: 15 - DNA, 20 bp, Artificial
  Synthetic
tgaggcactggagatcactg

SEQ ID NO: 16 - DNA, 20 bp, Artificial
  Synthetic
gggccataatctgcaaacag

SEQ ID NO: 17 - DNA, 20 bp, Artificial
  Synthetic
agccacatcgctcagacacc

SEQ ID NO: 18 - DNA, 19 bp, Artificial
  Synthetic
gtactcagcgccagcatcg

SEQ ID NO: 19 - DNA, 20 bp, Artificial
  Synthetic
gcgctcttcagatccacgag

SEQ ID NO: 20 - DNA, 21 bp, Artificial
  Synthetic
cccgtgtgtcatgtagtgaac

SEQ ID NO: 21 - DNA, 20 bp, Artificial
  Synthetic
gcagagtgttcctgaaggaa

SEQ ID NO: 22 - DNA, 25 bp, Artificial
  Synthetic
ctcaggtaaatcatctactttcagc

SEQ ID NO: 23 - DNA, 20 bp, Artificial
  Synthetic
gaggaacgaggtgtcgtttg

SEQ ID NO: 24 - DNA, 20 bp, Artificial
  Synthetic
cacgtggtaaacagatcaga

SEQ ID NO: 25 - DNA, 20 bp, Artificial
  Synthetic
gcactggcggtgtcaagccc

SEQ ID NO: 26 - DNA, 20 bp, Artificial
  Synthetic
ataagtggaccgtgagatgc

SEQ ID NO: 27 - DNA, 20 bp, Artificial
  Synthetic
acagccacacactacatcag

SEQ ID NO: 28 - DNA, 20 bp, Artificial
  Synthetic
ttgtgacgatctccaattcc

SEQ ID NO: 29 - DNA, 20 bp, Artificial
  Synthetic
gcagtttgctccggaatatg

SEQ ID NO: 30 - DNA, 20 bp, Artificial
  Synthetic
gctggccagcacagacgctg